(19) United States
(12) Patent Application Publication
(10) Pub. No.: US 2026/0061193 A1
(43) Pub. Date: Mar. 5, 2026

(54) IMPLANTABLE MEDICAL SYSTEMS, DEVICES, AND METHODS FOR BI-HEMISPHERIC STIMULATION OF THE DIAPHRAGM

(71) Applicant: VisCardia, Inc., Beaverton, OR (US)

(72) Inventor: Peter T. Bauer, Portland, OR (US)

(21) Appl. No.: 19/295,167

(22) Filed: Aug. 8, 2025

Related U.S. Application Data
(60) Provisional application No. 63/688,541, filed on Aug. 29, 2024.

(51) Int. Cl. A61N 1/36

(52) U.S. Cl. CPC A61N 1/3601

(57) ABSTRACT

Devices, systems and methods affect cardiac function of a patient by delivering a stimulation to a right hemisphere of a diaphragm of the patient during a diastolic phase of a cardiac cycle of the patient, wherein the stimulation results in an asymptomatic, transient, partial contraction of the right hemisphere of the diaphragm; and delivering a stimulation to the left hemisphere of the diaphragm during a systolic phase of the cardiac cycle, wherein the stimulation results in an asymptomatic, transient, partial contraction of the left hemisphere of the diaphragm.

### CROSS-REFERENCE TO RELATED APPLICATIONS

This application claims the benefit of U.S. Provisional Application Serial No. 63/688,541, entitled “Implantable Medical Systems, Devices, and Methods for Bi-Hemispheric Stimulation of the Diaphragm” and filed on August 29, 2024, which is expressly incorporated by reference herein in its entirety.

### TECHNICAL FIELD

The present disclosure relates generally to systems, devices and method for affecting cardiac function, and more particularly, to implantable medical systems, devices and methods that affect pressures within the intrathoracic cavity through bi-hemispheric stimulation of the diaphragm.

### BACKGROUND

The diaphragm is a dome shaped skeletal muscle structure separating the thoracic and abdominal cavities. It is the major muscular organ responsible for mechanical respiratory motion by deflecting downwards upon contraction during inspiration. The phrenic nerve innervates the diaphragm and acts as the primary method of nervous excitation to signal contraction. The external and internal intercostal muscles also elevate the ribs increasing the anterior-posterior diameter of the thoracic cavity. During inspiration, the movement of the diaphragm results in expansion and negative pressure within the thoracic cavity as the diaphragm and intercostal muscles increase the size of the thorax. The expanding thorax causes the intrathoracic pressure to decrease below atmospheric pressure and air moves into the lungs. During exhalation, the inspiratory muscles relax, and the elastic recoil of the lung tissues, combined with a rise in intrathoracic pressure, causes air to move out of the lungs.

Changes in intrathoracic pressure from diaphragmatic contraction and thoracic expansion may be transmitted to the intrathoracic structures namely the heart, pericardium, great arteries and veins. Spontaneous inspiration produces a negative pleural pressure affecting cardiovascular performance including atrial filling (preload) and resistance to ventricular emptying (afterload). This affect can be observed in cardiovascular hemodynamic parameters during normal function when diaphragmatic contractions are of sufficient duration, intensity and expansiveness to cause inspiration, and used in clinical practice during Vasalva and Mueller maneuvers where patients forcefully inspire or expire using diaphragmatic muscles against a closed glottis causing a rapid change in thoracic pressures. These maneuvers result in pronounced rapid acute changes to intrathoracic pressure, which changes in turn alter pressure gradients associated with the cardiac chambers and vessels to affect cardiac functions, including cardiac filling and output.

The effects of intrathoracic pressure on cardiac systemic performance are complex. Hiccups, which result from rapid partial diaphragmatic contractions causing rapid decreases to intrathoracic pressure, have been previously used to characterize their effects of cardiac and systemic performance. Studies of both animal and human subjects demonstrated changes to hemodynamic parameters including overall ventricular diastolic and systolic pressures, cardiac output and changes to systemic measures including aortic distention and vascular resistance. These studies also demonstrated that rapid intrathoracic pressure effect changes are extremely sensitive to timing relative to the cardiac cycle, with different observed if the hiccups occur during ventricular diastolic, systole, or during the diastole-systole transition.

### BRIEF DESCRIPTION OF THE DRAWINGS

Various aspects of systems, devices and methods that affect pressures within the intrathoracic cavity through diaphragmatic stimulation will now be presented in the detailed description by way of example, and not by way of limitation, referring to the accompanying drawings, wherein:

FIG. 1A is an illustration of structures for bi-hemispheric synchronized asymptomatic diaphragm stimulation (ADS) including a first ADS therapy delivery mechanism associated with the left hemisphere and a second ADS therapy delivery mechanism associated with the right hemisphere.

FIG. 1B is an illustration of the structures of FIG. 1A viewed from the inferior side of the diaphragm and in the caudal direction showing the first ADS therapy delivery mechanism associated with the left hemisphere and the second ADS therapy delivery mechanism associated with the right hemisphere.

FIG. 2A is an illustration of the thoracic cavity at end inspiration.

FIG. 2B is an illustration of the thoracic cavity at end expiration.

FIG. 3A is an illustration an implantable medical device (IMD) for providing bi-hemispheric synchronized ADS therapy, including a first ADS therapy delivery mechanism coupled to a diaphragm lead and a second ADS therapy delivery mechanism coupled to a diaphragm lead.

FIG. 3B is an illustration of the IMD of FIG. 3A implanted with the first ADS therapy delivery mechanism placed on the superior side of the left hemisphere and the second ADS therapy delivery mechanism placed on the superior side of the right hemisphere.

FIG. 3C is an illustration of the IMD of FIG. 3A implanted with the first ADS therapy delivery mechanism placed on the inferior side of the left hemisphere and the second ADS therapy delivery mechanism placed on the inferior side of the right hemisphere.

FIG. 4A is an illustration of an IMD for providing bi-hemispheric synchronized ADS therapy and another therapy, such as cardiac rhythm management (CRM) therapy, including a first ADS therapy delivery mechanism coupled to a diaphragm lead and a second ADS therapy delivery mechanism coupled to a diaphragm lead.

FIG. 4B is an illustration of the IMD of FIG. 4A implanted with a first ADS therapy delivery mechanism placed on the superior side of the left hemisphere and a second ADS therapy delivery mechanism placed on the superior side of the right hemisphere.

FIG. 5 is an illustration of an implantable medical system for providing bi-hemispheric synchronized ADS therapy, including a first ADS therapy delivery mechanism associated with a first diaphragm IMD and a second ADS therapy delivery mechanism associated with a second diaphragm IMD.

FIG. 6 is an illustration of the system of FIG. 5 implanted with the first diaphragm IMD placed on the superior side of the left hemisphere and the second diaphragm IMD placed on the superior side of the right hemisphere.

FIG. 7 is a block diagram of an implantable medical device configured to provide bi-hemispheric synchronized ADS.

FIG. 8A is a diagram of bi-hemispheric synchronized ADS, where a first stimulation is delivered to the right diaphragm during diastole of a cardiac cycle and a second stimulation is delivered during systole.

FIG. 8B are illustrations of an electrocardiogram (ECG) waveform and a diaphragmatic acceleration waveform including spaced apart pairs of transient, partial contractions of the diaphragm resulting from delivery of the bi-hemispheric synchronized ADS of FIG. 8A.

FIG. 9 is a flowchart of a method of delivering bi-hemispheric synchronized ADS.

FIG. 10 are illustrations of an ECG waveform and a diaphragmatic acceleration waveform including a series of transient, partial contractions, each having a caudal phase followed by an extended or enhance cranial phase that results from delivery of a closely timed pair of stimulation pulses to a hemisphere of the diaphragm.

### SUMMARY

Devices, systems and methods affect cardiac function of a patient by delivering a stimulation to a right hemisphere of a diaphragm of the patient during a diastolic phase of a cardiac cycle of the patient, wherein the stimulation results in an asymptomatic, transient, partial contraction of the right hemisphere of the diaphragm; and delivering a stimulation to the left hemisphere of the diaphragm during a systolic phase of the cardiac cycle, wherein the stimulation results in an asymptomatic, transient, partial contraction of the left hemisphere of the diaphragm.

It is understood that other aspects of apparatuses and methods will become readily apparent to those skilled in the art from the following detailed description, wherein various aspects of apparatuses and methods are shown and described by way of illustration. As will be realized, these aspects may be implemented in other and different forms and its several details are capable of modification in various other respects. Accordingly, the drawings and detailed description are to be regarded as illustrative in nature and not as restrictive.

### DETAILED DESCRIPTION

An objective of the devices, systems and methods disclosed herein is to optimize numerous various and interdependent physiologic effects between the heart and vessels as a means of managing heart failure and improving the patient’s overall condition. To this end, evoked transient partial diaphragmatic contractions are used to optimize the operating intrathoracic pressure conditions on the heart and vessels and other interdependent physiologic effects. These interdependent physiologic effects include: the blood volume to one or more chambers of the cardiovascular system within the thoracic cavity, end diastolic pressure (preload) that causes changes to systolic output (starling), that mediates intracardiac blood flow (diastolic coronary perfusion) and operating mechanics (efficiency), or for decreasing the compliance of the vessels responsible for cardiac filling (vena cava and right atrium) or for altering the compliance of cardiac vessels to better match the operational ability of the heart (impedance matching or optimization). These indirect physiologic mechanisms will augment the direct physiologic mechanism of mechanically augmenting the mechanical forces of the heart and decreasing the vascular resistance to cardiac output.

### Thoracic Cavity

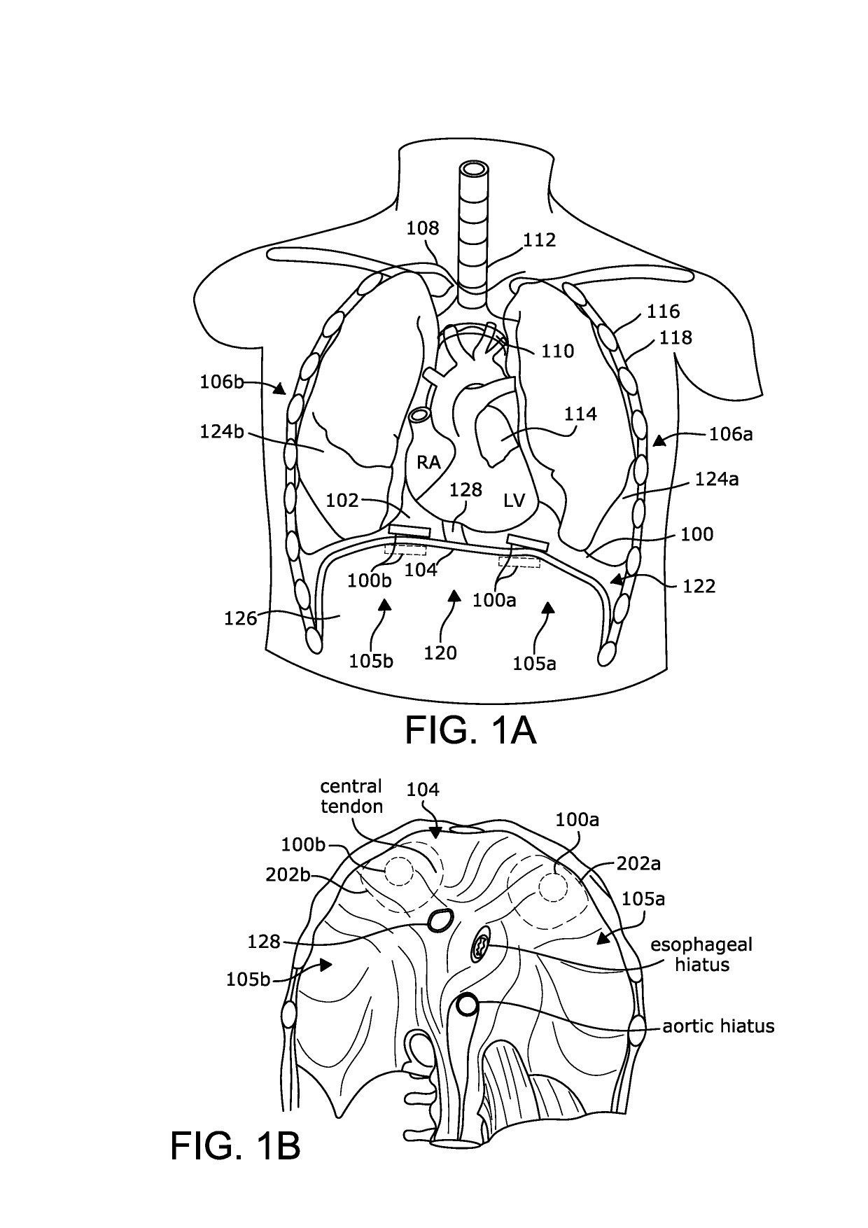

With reference to FIGS. 1A and 1B, the thoracic cavity 102, also referred to as the intrathoracic cavity and the mediastinum, is a hermetically sealed cavity formed by various connected structures. These structures include the diaphragm 104, the thoracic sidewalls 106a, 106b, and layered walls 108, 110, near the trachea 112 and the heart 114. The thoracic sidewalls 106a, 106b are formed of ribs 116 and membrane 118 filing the space between the ribs, and define the thoracic sidewalls 106a, 106b of the thoracic cavity 102. The layered walls 108, 110 are formed of various membranes and vessels which lay over each other to form a seal at the top of the thoracic cavity 102.

The diaphragm 104 is a dome-shaped skeletal muscle structure located below the lungs 124a, 124b that separates the thoracic cavity 102 from the abdominal cavity 126. The diaphragm 104 defines the lower end of the thoracic cavity 102 and is the major muscular organ responsible for mechanical respiratory motion. The diaphragm 104 includes a left hemisphere 105a and a right hemisphere 105b. The left and right hemispheres 105a, 105b are electrically isolated from each other, and their locations are adjacent to different parts of the cardiovascular anatomy in the chest or abdomen. More specifically, the left hemisphere 105a is adjacent to the apex and the left ventricle (LV) of the heart 114, while the right hemisphere 105b is adjacent to the right atrium (RA) of the heart and the vena cava 128.

Mechanical respiratory motion includes an inspiration or inhalation phase and an expiration or exhalation phase. The phrenic nerve (not shown) innervates the diaphragm 104 and sends signals to the diaphragm to control inspiration and expiration. These signals act as the primary mechanism for initiating contraction of the diaphragm through nervous excitation. Since nervous endings responsible for pain sensation are absent within the diaphragm, a confine of therapy outputs are those which provide the desired hemodynamic effects to the cardiovascular system while simultaneously minimizing the likelihood of field stimulation of pain nerves contained within other nearby innervated thoracic cavity musculature.

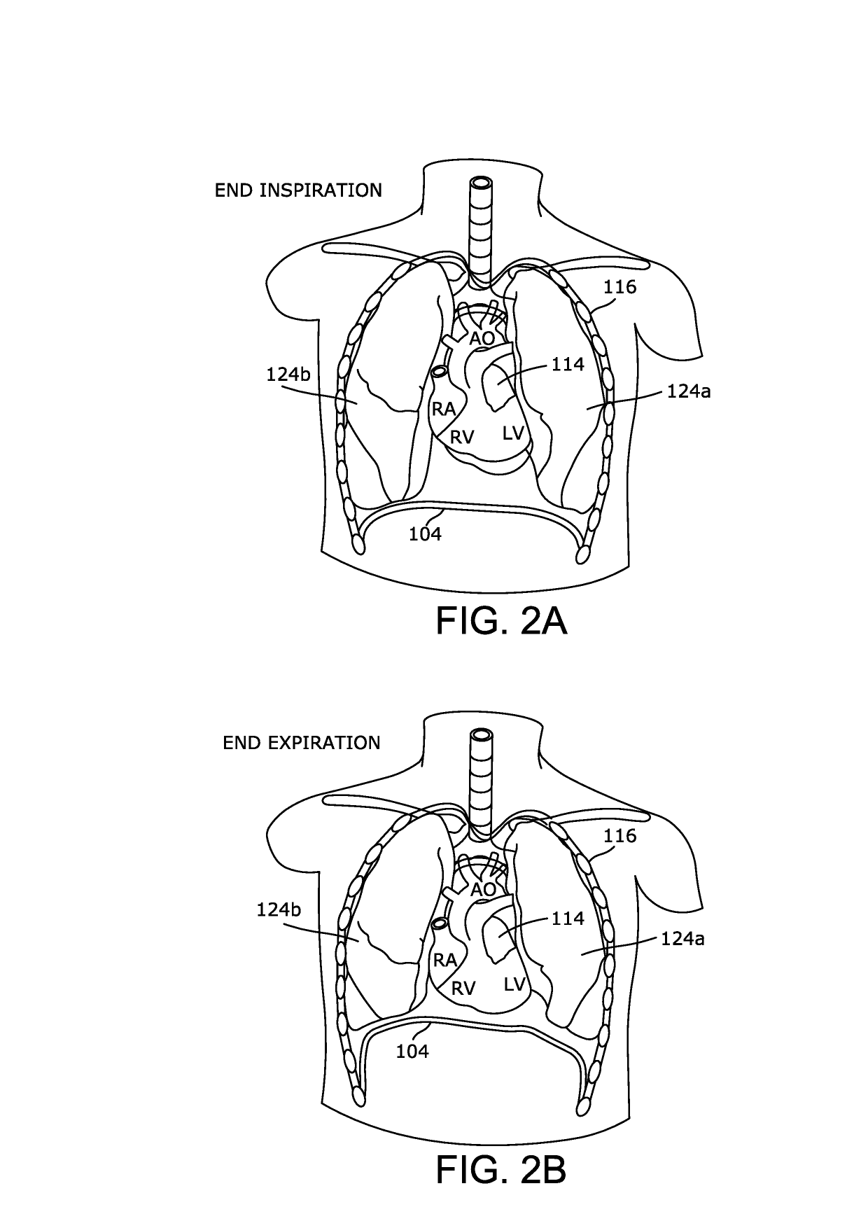

FIG. 2A is an illustration of the thoracic cavity at end inspiration. During inspiration, the diaphragm 104 contracts, e.g., flattens out, and deflects downward, in a direction away from the lungs 124a, 124b. Concurrent with downward deflection of the diaphragm during inspiration, the external and internal intercostal muscles around the lungs 124a, 124b elevate the ribs 116, thereby increasing the anterior-posterior diameter of the thoracic cavity 102. During inspiration, the movement of the diaphragm 104 results in expansion and negative pressure within the thoracic cavity 102 as the diaphragm and intercostal muscles increase the size of the thorax. The expanding thorax causes the pressure within the open space of thoracic cavity 102, i.e., the intrathoracic pressure, to decrease below atmospheric pressure. The pressure decrease causes external air to move into the lungs 124a, 124b.

FIG. 2B is an illustration of the thoracic cavity at end expiration. During expiration, the diaphragm 104 expands, e.g., assumes a dome shape, and deflects upward, in the direction of the lungs 124a, 124b. During expiration, the diaphragm 104, together with the external and internal intercostal muscles around the lungs 124a, 124b relax. The diaphragm 104 expands, e.g., resumes a dome shape, and the ribs 116 de-elevate, thereby reducing the anterior-posterior diameter of the thoracic cavity 102, and causing the intrathoracic pressure to increase above atmospheric pressure. The increase in intrathoracic pressure in combination with the elastic recoil of lung tissues, causes air to move out of the lungs.

Changes in the pressure within the open space of the thoracic cavity 102, i.e., the intrathoracic pressure, due to diaphragm contraction and thoracic cavity expansion, and diaphragm expansion and thoracic cavity contraction bring about changes in other pressures within the intrathoracic cavity, including pressures associated with intrathoracic structures like the heart 114, pericardium, great arteries and veins. For example, changes in cardiovascular pressures, such as right atrial (RA) pressure, right ventricular (RV) pressure, left ventricular (LV) pressure, and aortic (AO) pressure result from changes in intrathoracic pressure.

### Bi-Hemispheric Synchronized Diaphragmatic Stimulation

With reference to FIGS. 1A and 1B, in accordance with presently disclosed embodiments, intrathoracic pressure is manipulated through controlled delivery of diaphragmatic stimulation through a pair of ADS therapy delivery mechanisms 1001, 100b, to bring about desirable changes in other pressures within the intrathoracic cavity to improve cardiac function. Through delivery of appropriately timed diaphragm stimulations to each of the left hemisphere 105a and the right hemisphere 105b of the diaphragm 104, respective transient, asymptomatic, partial contractions of the left hemisphere and the right hemisphere are induced in synchrony or near synchrony with one or more cardiac events to deliver ADS therapy at specified portions of a cardiac cycle. As the left hemisphere 105a and the right hemisphere 105b of the diaphragm 104 are electrically isolated from each other, and their locations are adjacent to different parts of the cardiovascular anatomy in the chest or abdomen, a stimulation of both the left hemisphere and the right hemisphere of the diaphragm at the same or different time intervals from events in the cardiac cycle has more impact on cardiac performance than just a stimulation to a single hemisphere.

As used herein, a “transient” contraction of the diaphragm 104 is a short, twitching, caudal followed by cranial motion of the diaphragm that lasts in range of 60 to 180 msec., and is typically about 100 msec. A “partial” contraction of the diaphragm 104 is the part or region of the diaphragm (less than the entirety of the diaphragm) that exhibits a “transient” contraction. For example, with reference to FIG. 1B, a diaphragm stimulation delivered through an ADS therapy delivery mechanism 100a placed on the left hemisphere 105a of a diaphragm 104 will result in contraction of a portion 202a or part of the left hemisphere that is less than the entirety of the left hemisphere. A diaphragm stimulation delivered through an ADS therapy delivery mechanism 100b placed on the right hemisphere 105b of a diaphragm 104 will result in contraction of a portion 202b or part of the right hemisphere that is less than the entirety of the right hemisphere.

Timing the occurrences of these transient, asymptomatic, partial contractions relative to cardiac events results in changes in intrathoracic pressure, which in turn, increases and/or decreases pressures associated with the heart, pericardium, great arteries and veins to thereby improve hemodynamic function of the heart and manage heart failure. In some embodiments, a noted improvement in cardiac function can be achieved by stimulating the right hemisphere 105b at the end of diastole, and then stimulating the left hemisphere 105a more toward the beginning of systole.

In an example of this embodiment, a diaphragm stimulation to the left hemisphere 105a is synchronized with, or otherwise timed to an occurrence of a cyclic cardiac event, such as ventricular systole. This left-hemisphere stimulation may accelerate negative intrathoracic cavity pressure (suction) during ventricular filling to increase filling volume, and then accelerate positive intrathoracic cavity pressure (compression) to augment systolic contractile forces generated by the left ventricle. The left-hemisphere stimulation captures the area below and adjacent to the heart apex and left ventricle, and the resulting localized reduction in intra-thoracic pressure leads to a reduction in pressure on the heart (i.e., pericardial pressure) and pressure on the aorta on that side.

A diaphragm stimulation to the right hemisphere 105b is synchronized with, or otherwise timed to an occurrence of a cyclic cardiac event, such as ventricular diastole. Stimulating the right hemisphere 105b from a location closer to the vena cava 128 has more impact on the venous blood return into the heart, and that blood return at the right time in the cardiac cycle improves the so-called preload reserve and thereby cardiac function. This right hemisphere stimulation may accelerate negative intrathoracic cavity pressure (suction) during ventricular filling to increase filling volume, and then accelerate positive intrathoracic cavity pressure (compression) to augment diastolic flow generated by the compression of the vena cava 128. The right-hemisphere stimulation captures the area below and adjacent to the vena cava 128. The resulting localized reduction in intra-thoracic pressure leads to a reduction in pressure on the vena cava 128 and an increased filling volume of the heart chambers during the caudal period of the ADS induced “transient” motion of the diaphragm, while the subsequent cranial motion leads to an increased pressure on the vena cava 128 and therefore a further increase in filling volume of the heart chambers.

FIGS. 1A and 1B are illustrations of a device or system including a first asymptomatic diaphragmatic stimulation (ADS) therapy delivery mechanism 100a implanted in the region of a thoracic cavity 102 on or near the left hemisphere of the diaphragm 104, and a second ADS therapy delivery mechanism 100b implanted in the region of the thoracic cavity 102 on or near the right hemisphere of the patient's diaphragm. Each of the first and second ADS therapy delivery mechanisms 100a, 100b is configured to deliver a stimulation to the diaphragm 104 in accordance with a diaphragm stimulation program.

The ADS therapy delivery mechanisms 100a, 100b may include one or more electrodes configured to be positioned on or near a diaphragm, and to deliver electrical stimulation pulses to the diaphragm. The ADS therapy delivery mechanisms 100a, 100b may include a mechanical transducer configured to be positioned on or near a diaphragm, and to deliver mechanical stimulation to the diaphragm. As described later in this disclosure, the ADS therapy delivery mechanisms 100a, 100b can be included in a lead of an implantable medical device (IMD), or in one or multiple single-piece, unitary structures, e.g., leadless IMDs. In either configuration, the IMD includes a controller that implements the diaphragm stimulation program. Details on the controller are provided later in this disclosure.

Continuing with FIGS. 1A and 1B, the first and second ADS therapy delivery mechanisms 100a, 100b may be placed, through conventional thoracotomy, thoracoscopy, or through a subxiphoid surgical access at a selected surface region of the diaphragm 104 on the superior side of diaphragm 104 at a location referred to as a superior implant location 122. For example, the first ADS therapy delivery mechanism 100a may be positioned between the superior surface of diaphragm 104 and the underside of the left lung 124a and in proximity of the apex of the heart 114 and left ventricular side. For example, the second ADS therapy delivery mechanism 100b may be positioned between the superior surface of diaphragm 104 and the right lung 124b and in proximity of the right side of the heart, in an area near the vena cava 128.

Alternatively, the first and second ADS therapy delivery mechanisms 100a, 100b may be placed, through conventional laparoscopy or laparotomy, at a selected surface region of the diaphragm 104 on the inferior side of the diaphragm at a location referred to as an inferior implant location 120. For example, the first ADS therapy delivery mechanism 100a may be positioned on the inferior side of the left hemisphere 105a in proximity of the apex of the heart 114 and the left ventricular side. For example, the second ADS therapy delivery mechanism 100b may be positioned on the inferior side of the right hemisphere 105b in proximity of the right side of the heart, in an area near the vena cava 128.

### Leaded Implantable Medical Device

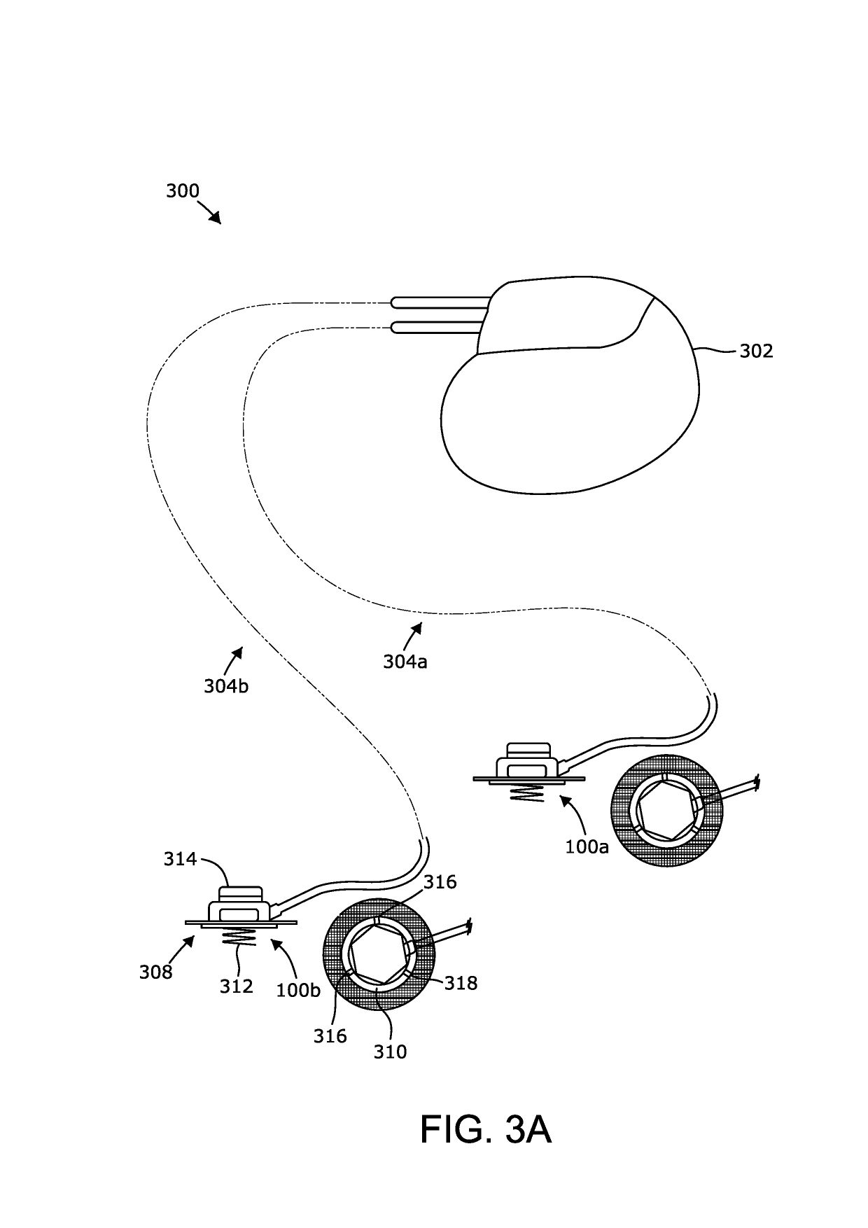

With reference to FIGS. 3A, 3B, and 3C, the ADS therapy delivery mechanisms 100a, 100b may be included in a leaded IMD 300 that only delivers bi-hemispheric ADS therapy. The leaded IMD 300 includes a can or housing 302 that houses a controller, and a pair of diaphragm leads 304a, 304b that mechanically couple to the housing and electrically couple to the controller in the housing. The controller is configured to generate asymptomatic stimulation pulses for stimulating the diaphragm 104 through the diaphragm leads 304a, 304b. Each lead 304a, 304b has an ADS therapy delivery mechanism 100a, 100b at its distal end 308. In the configuration of FIG. 3A, each ADS therapy delivery mechanism 100a, 100b comprises a ring electrode 310 and a helix electrode 312. The leads 304a, 304b is configured for implant on a surface of a biological membrane, e.g., diaphragm, forming part of a hermetically sealed biological cavity.

Each lead 304a, 304b includes a sensor assembly 314 at its distal end 308. The sensor assembly 314 includes one or more sensors 316, 318. The sensors may be, for example, electrodes 316 for sensing cardiac electrical activity, or a motion sensor 318, e.g., an acoustic transducer for sensing heart sounds, or an accelerometer for sensing mechanical motion of the heart and/or the diaphragm. The electrodes 316 and motion sensor 318 may be spaced apart around the circumference of the ring electrode 310 and electrically isolated therefrom.

With reference to FIG. 3B, in some cases the housing 302 may be implanted subcutaneously in a surgically created pocket in the infraclavicular pectoral region or at the side of the chest below the armpit. The ADS therapy delivery mechanism 100a portion of the diaphragm lead 304a may be implanted on the superior side of the left hemisphere 105a of the diaphragm through conventional thoracotomy, thoracoscopy, or through a subxiphoid surgical access. The ADS therapy delivery mechanism 100b portion of the diaphragm lead 304b, may be implanted on the superior side of the right hemisphere 105b of the diaphragm through conventional thoracotomy, thoracoscopy, or through a subxiphoid surgical access.

With reference to FIG. 3C, in some cases the housing 302 may be implanted subcutaneously in a surgically created pocket in the anterior or lateral lumbar region. The ADS therapy delivery mechanism 100a portion of the diaphragm lead 304a may be implanted on the inferior side of the left hemisphere 105a of the diaphragm through conventional laparoscopy or laparotomy. The ADS therapy delivery mechanism 100b portion of the diaphragm lead 304b, may be implanted on the inferior side of the right hemisphere 105b of the diaphragm through conventional laparoscopy or laparotomy.

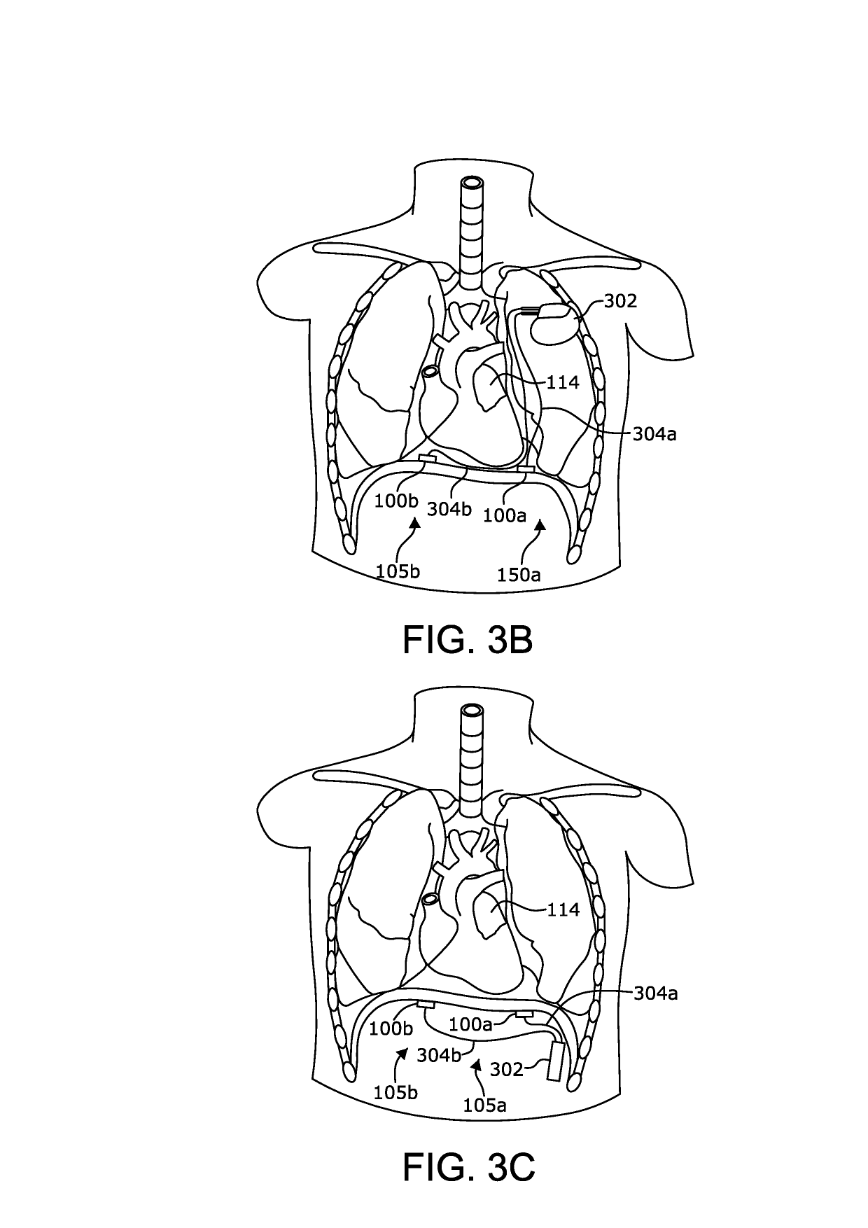

With reference to FIGS. 4A and 4B, the ADS therapy delivery mechanisms 100a, 100b may be included in a leaded IMD 400 that delivers bi-hemispheric ADS therapy and another therapy, such as a cardiac rhythm management (CRM) therapy, e.g., pacing, cardiac resynchronization, or defibrillation. This leaded IMD 400 includes a housing 502 that houses a controller, one or more cardiac leads 504, 506 that support pacemaker functionality, defibrillation functionality, or both, and a pair of diaphragm stimulation leads 408a, 408rb. The pair of diaphragmatic stimulation leads 408a, 408b support ADS therapy and may be configured the same as the leads 304a, 304b describe above with reference to FIG. 3.

The controller is configured to generate pacing pulses for pacing the heart 114 through one or more of the cardiac leads 504, 506, generate defibrillation energy pulses for defibrillating the heart through a cardiac lead 504, and generate asymptomatic stimulation pulses for stimulating the diaphragm 104 through the diaphragm stimulation leads 408a, 408b. The controller and diaphragmatic stimulation leads 408a, 408b may include electrical and mechanico-electrical componentry to perform cardiac sensing functionality for purposes of cardiac synchronized diaphragmatic stimulation. Alternatively, in the case of a pacemaker, one or more of the cardiac leads 504, 506 may perform cardiac sensing functionality for purposes of cardiac synchronized diaphragmatic stimulation.

The housing 502 may be implanted subcutaneously in a surgically created pocket at an infraclavicular pectoral region in accordance with standard pacemaker and/or cardioverter/defibrillator implant procedures. Each of the cardiac leads 504, 506 is configured to be implanted into the heart through the subclavian vein. For example, a pacing lead 506 may terminate in the right atrium, while a defibrillator lead 504 extends into the right ventricle.

The ADS therapy delivery mechanism 100a portion of the diaphragm lead 304a may be implanted on the superior side of the left hemisphere 105a of the diaphragm 104 through conventional thoracoscopy or thoracotomy accessed near the infraclavicular pocket, or through a sub-xiphoid approach by creating a subcutaneous tunnel from the location of the housing 502 parallel to the sternum until reaching a sub sternal location from where a laparoscopic thoracotomy is performed at a subxiphoid location to reach the superior region of the left hemisphere of diaphragm.

Similarly, the ADS therapy delivery mechanism 100b portion of the diaphragm lead 304b, may be implanted on the superior side of the right hemisphere 105b of the diaphragm 104 through conventional thoracotomy accessed near the infraclavicular pocket, or through a sub-xiphoid approach by creating a subcutaneous tunnel from the location of the housing 502 parallel to the sternum until reaching a sub sternal location from where a laparoscopic thoracotomy is performed at a subxiphoid location to reach the superior region of the right hemisphere of diaphragm.

### Leadless Implantable Medical System

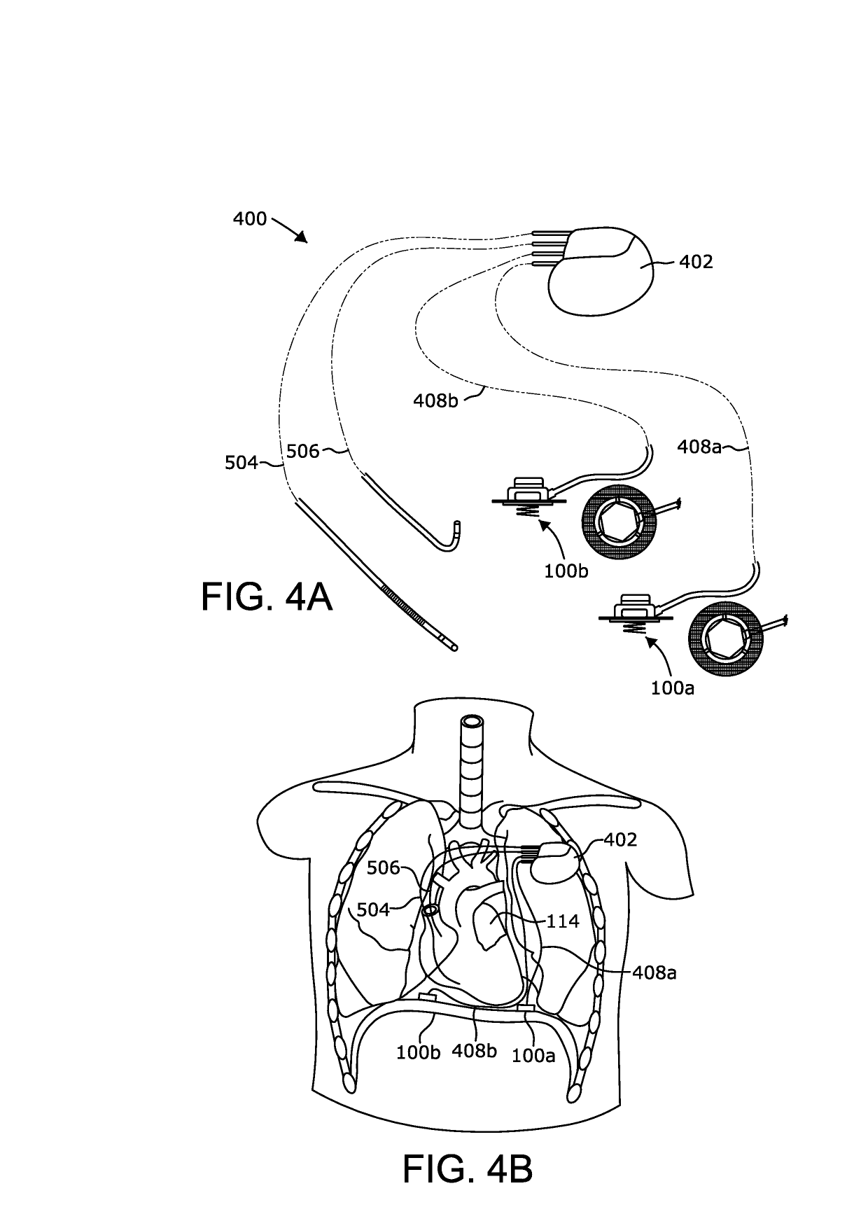

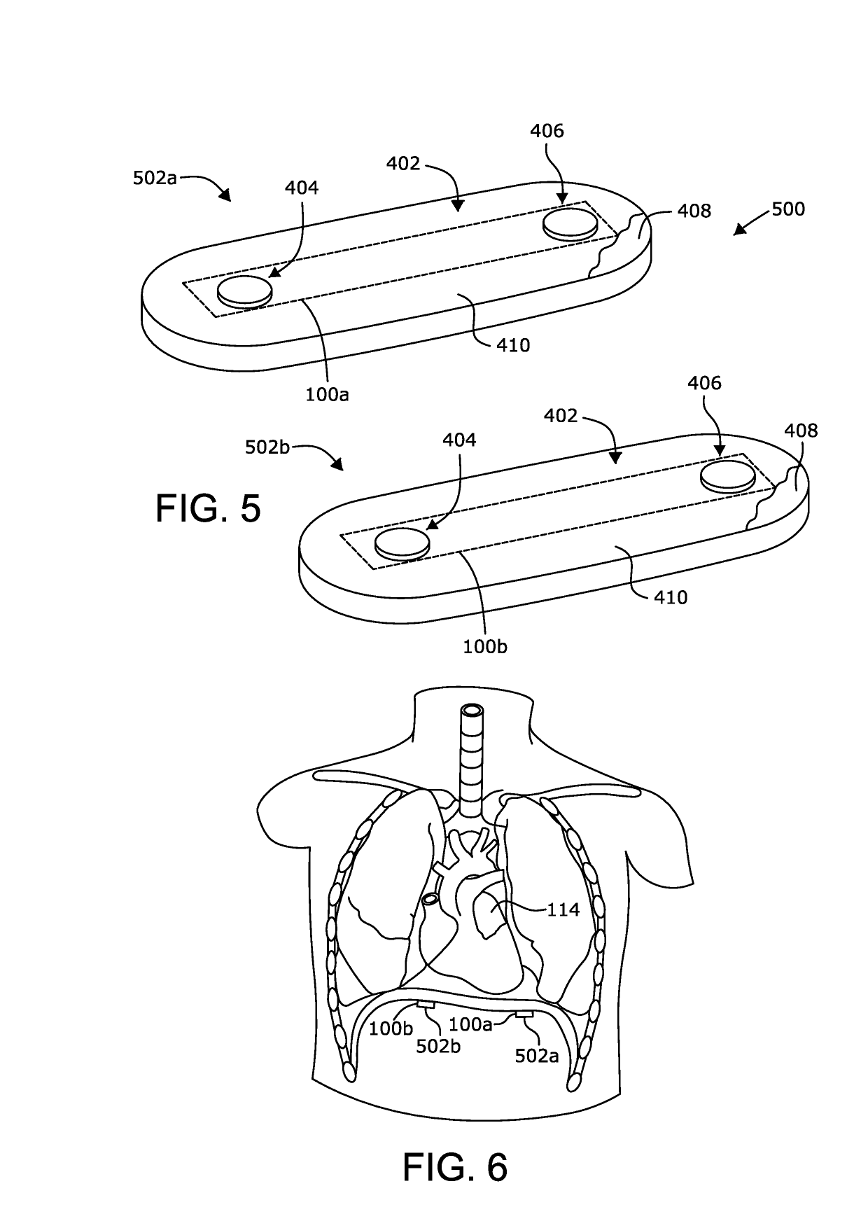

With reference to FIGS. 5 and 6, the ADS therapy delivery mechanisms 100a, 100b may be included in a leadless implantable medical system 500 that delivers only bi-hemispheric ADS therapy. The leadless implantable medical system 500 includes a first leadless diaphragm IMDs 502a and a second leadless diaphragm IMD 502b, each having a housing 402 with at least two electrodes 404, 406 associated with a surface of the housing. The two electrodes 404, 406 form the ADS therapy delivery mechanisms 100a, 100b.

While the leadless diaphragm IMDs 502a, 502b illustrated in FIG. 5 are formed is the shape of an elongated disk, the IMD may have other form factors, including for example, a tube. The leadless diaphragm IMDs 502a, 502b may have a length of about 1.25-inches, a width of about 0.5-inches, and a thickness of about 0.125-inches. The two electrodes 404, 406 are spaced apart by about 1-inch and are located on a surface 408 of the housing. A non-electrically-conductive, biocompatible mesh 410 may be affixed to the surface 408 to facilitate anatomical bonding of the leadless implantable medical system 500 to the surface region of the diaphragm 104.

The leadless diaphragm IMDs 502a, 502b are configured for implant on a surface of a biological membrane, e.g., a diaphragm 104.

The first and second leadless diaphragm IMDs 502a, 502b may be placed, through conventional thoracotomy or even thoracoscopy, at a selected surface region of the diaphragm 104 on the superior side of diaphragm 104 at a location referred to as a superior implant location 122. For example, the first leadless diaphragm IMDs 502a may be positioned between the superior surface of diaphragm 104 and the underside of the left lung 124a. For example, the second leadless diaphragm IMDs 502b may be positioned between the superior surface of diaphragm 104 and the right side of the heart 114, in an area near the vena cava 128. Alternatively, the first and second leadless diaphragm IMDs 502a, 502b may be placed, through conventional laparoscopy, at a selected surface region of the diaphragm 104 on the inferior side of the diaphragm at a location referred to as an inferior implant location 120.

### Implantable Medical Devices with ADS Therapy

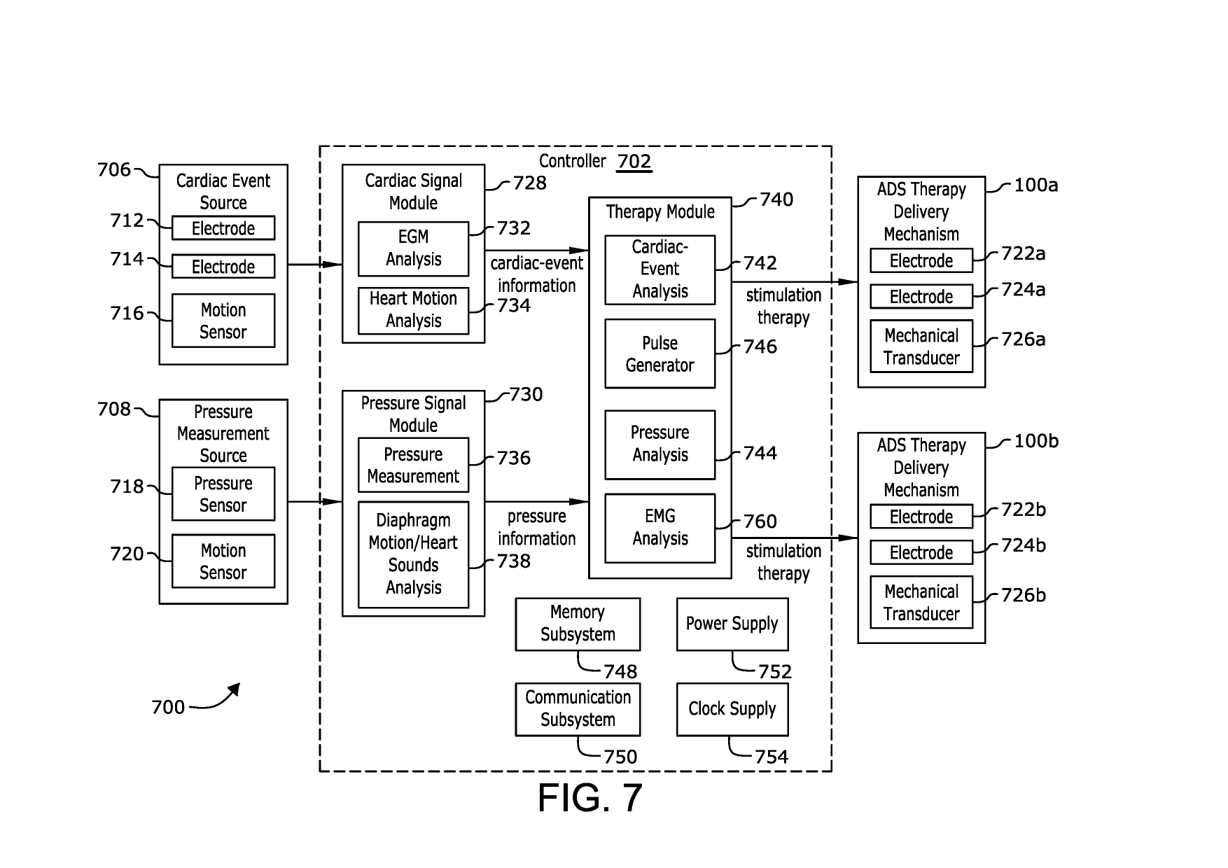

FIG. 7 is a block diagram of an IMD 700 configured to affect pressures within the intrathoracic cavity through delivery of bi-hemispheric ADS therapy. The IMD 700 includes a controller 702, a cardiac event source 706, a pressure measurement source 708, a first ADS therapy delivery mechanism 100a, and a second ADS therapy delivery mechanism 100b, each of which may be coupled for interaction with the controller, either through a wired connection or through a wireless connection. The controller 702 includes a cardiac signal module 728, a pressure signal module 730, a therapy module 740, and various other modules.

The cardiac event source 706 is configured to provide signals to the controller 702 that represent cardiac events. For example, the cardiac event source 706 may be one or more electrodes 712, 714 configured to be positioned on or near a diaphragm to sense electrical signals representative of cardiac events and to provide the signals to the controller 702. Alternatively, the one or more electrodes 712, 714 may be configured to be positioned in, on, or adjacent to an intrathoracic structure, e.g., heart, pericardium, great artery and vein, within the intrathoracic cavity. In this case, the one or more electrodes 712, 714 may be associated with a device configured to be implanted remote from the controller 702 and to provide signals sensed by the electrodes to the controller through a wireless communication link.

The cardiac event source 706 may also be a motion sensor 716 configured to be positioned on or near a diaphragm to sense motion of the heart or to sense heart sounds, and to output electrical signals representative of such motion. Alternatively, the motion sensor 716 may be configured to be positioned in, on, or adjacent to an intrathoracic structure, e.g., heart, pericardium, great artery and vein, within the intrathoracic cavity. In this case, the motion sensor 716 may be associated with a device configured to be implanted remote from the controller 702 and to provide signals sensed by the motion sensor to the controller through a wireless communication link. In either case, the motion sensor 716 may be, for example, an accelerometer (such as a multi-axial e.g., three-dimensional, accelerometer) that provides signal related to heart movement, or an acoustic transducer that provides signal related to heart sounds.

The pressure measurement source 708 is configured to provide signals to the controller 702 that represent one or more pressures within the intrathoracic cavity. “Pressures within the intrathoracic cavity” may include an intrathoracic pressure obtained directly through a pressure sensor placed in the open space of the intrathoracic cavity and outside of any intrathoracic structures, e.g., heart, pericardium, great arteries and veins, within the cavity. “Pressures within the intrathoracic cavity” may also include a measure of intrathoracic pressure obtained indirectly, for example, through an accelerometer placed outside of the intrathoracic cavity that provides a measure indicative of, or correlated with, intrathoracic pressure. “Pressures within the intrathoracic cavity” may also include pressures associated with intrathoracic structures like the heart, pericardium, great arteries and veins. For example, these “pressures within the intrathoracic cavity” may include right atrial pressure, right ventricular pressure, left ventricular pressure, and aortic pressure.

The pressure measurement source 708 may be one or more pressure sensors 718 configured to be positioned in the open space of the intrathoracic cavity, or in, on, or adjacent an intrathoracic structure, e.g., heart, pericardium, great artery and vein, within the cavity, and configured to output electrical signals representative of pressure. To these ends, the one or more pressure sensors 718 may be directly coupled to the controller 702, or alternatively, associated with a device configured to be implanted remote from the controller 702 and to provide signals sensed by the one or more pressure sensors to the controller through a wireless communication link.

Direct coupling between the one or more pressure sensors 718 and the controller 702 may be appropriate when the IMD 700 is implanted on the superior side of the patient’s diaphragm at a superior implant location 122, such as shown in FIG. 1. When implanted in this location, pressure sensors 718 directly coupled to the controller 702 would be placed in the open space of the thoracic cavity 102. Remote coupling between the one or more pressure sensors 718 and the controller 702 may be appropriate when the IMD 700 is implanted on the inferior side of the patient’s diaphragm at an inferior implant location 120. When implanted in this location, one or more pressure sensors 718 separately implanted in the intrathoracic cavity and remotely coupled to the controller 702 may provide pressure signals. For example, the pressure sensor 718 may be included in a device configured to be implanted: 1) in the right atrium to obtain right-atrial pressure signals, 2) in the right ventricle to obtain right ventricular pressures, 3) in the right ventricle to obtain surrogates of pulmonary artery pressure, or 4) within the pulmonary artery itself.

The pressure measurement source 708 may also be a motion sensor 720 configured to provides signals indicative of, or that correlate to, intrathoracic pressure. For example, the motion sensor 720 may be an accelerometer configured to be positioned on or near a diaphragm to sense motion of the diaphragm, and to output electrical signals representative of such motion to the controller 702. As will be described further below, fluctuations in these electrical signals correlate to changes in intrathoracic pressure associated with respiration cycles. The motion sensor 720 may also be an accelerometer or acoustic transducer configured to be positioned within the patient to sense sounds associated with cardiac function, and to output electrical signals representative of such sounds. Fluctuations in these electrical signals correlate to changes in intrathoracic pressure associated with respiration cycles. Alternatively, the motion sensor 720 may be an impedance/conductance sensor in the form of a pair of electrodes configured to be positioned in or on the diaphragm, and to output electrical signals representative of impedance or conductance of diaphragm tissue. Fluctuations in impedance or conductance correlate to changes in expansion and contraction of the diaphragm, which in turn correlate to changes in intrathoracic pressure associated with respiration cycles.

The ADS therapy delivery mechanisms 100a, 100b are configured to apply stimulation to the diaphragm to cause asymptomatic, transient, partial contraction of the diaphragm. As previously mentioned, a “transient” contraction of the diaphragm is a short, twitching, caudal followed by cranial motion of the diaphragm that lasts in range of 60 to 180 msec., and is typically about 100 msec. A “partial” contraction of the diaphragm is the part or portion of the diaphragm (less than the entirety of the diaphragm) that exhibits a “transient” contraction. The stimulation is characterized by a set of stimulation parameters that induce a partial contraction of the diaphragm that does not affect respiration. More specifically, the stimulation is configured such that the diaphragm does not contract to a level that induces inspiration. The ADS therapy delivery mechanisms 100a, 100b may be one or more electrodes 722a, 724a, 722b, 724b configured to be positioned on or near a diaphragm to deliver electrical stimulation pulses to the diaphragm.

Considering the controller 702 in more detail, the cardiac signal module 728 of the controller receives signals from the cardiac event source 706 and is configured to process the signals to detect cardiac events of interest. For example, as will be described further below, the cardiac signal module 728 may be configured to detect one or more of an electrical cardiac event, such as a ventricular depolarization represented by an R-wave, and 2) a mechanical cardiac event, such as a ventricular contraction represented by an S1 heart sound. Information corresponding to detected cardiac events is provided to the therapy module 740, which in turn processes the cardiac-event information to determine or adjust one or more parameters of a stimulation therapy.

With respect to electrical cardiac events, the cardiac signal module 728 may include an electrogram (EGM) analysis module 732 adapted to receive electrical signals from the electrodes 712, 714 and to process the electrical signals to detect cardiac events of interest. The EGM analysis module 732 may be configured to process a cardiac electrical activity signal, e.g., an EGM signal, to detect cardiac events corresponding to atrial events, such as P waves, or ventricular events, such as R waves, QRS complexes, or T waves.

Regarding mechanical cardiac events, the cardiac signal module 728 may include a heart motion/sounds analysis module 734 for analyzing mechanical motion of the heart. The heart motion/sounds analysis module 734 is adapted to receive signals from the motion sensor 716 and to detect a cardiac event of interest. As previously mentioned, the motion sensor 716 may be, for example, an accelerometer or acoustic transducer, configured to sense a variety of mechanical and sound activities, such as diaphragm motion and heart sounds. Heart sound signals obtained through the accelerometer may be processed by the heart motion/sounds analysis module 734 to detect cardiac events.

The pressure signal module 730 of the controller 702 receives signals from the pressure measurement source 708 and is configured to process the signals for purposes of detecting a pressure event of interest or deriving a pressure measure of interest. For example, regarding measures of interest, the pressure signal module 730 may process signals from a pressure sensor 718 to determine pressure measurements under different therapy conditions, e.g., with diaphragmatic stimulation on, and with diaphragmatic stimulation off, or under different stimulation settings. The pressure signal module 730 may also process signals from a pressure sensor 718 to determine pressure measurements at different times, e.g., at or near delivery of a stimulation pulse, and at or near an occurrence of a particular cardiac event. Regarding events of interest, the pressure signal module 730 may process signals from a motion sensor 720 to detect respiration cycles and to identify one or more events of interest within the cycle, such as end inspiration. Information corresponding to detected events of interest and measures of interest, collectively referred to as pressure information, is provided to the therapy module 740. The therapy module 740, in turn, processes the pressure information to determine whether an adjustment to one or more parameters of a stimulation therapy is warranted.

Regarding the processing of signals from a pressure sensor 718, the pressure signal module 730 may include a pressure measurement module 736 for analyzing pressures within the intrathoracic cavity. The pressure measurement module 736 is adapted to receive signals from the pressure sensor 718. As previously described, the pressure sensor 718 may be a configured to be placed in the open space of the intrathoracic cavity and outside of any intrathoracic structures, e.g., heart, pericardium, great arteries and veins, within the cavity – to thereby provide a signal representing intrathoracic pressure. Alternatively, the pressure sensor 718 may be configured to be placed in, on, or adjacent an intrathoracic structure, e.g., heart, pericardium, great artery and vein, within the cavity. For example, the pressure sensor 718 may be configured to be placed in, on, or adjacent to one of the right atrium, the right ventricle, the left ventricle, the aorta, and the pulmonary artery – to thereby provide a corresponding signal presenting right atrial pressure, right ventricular pressure, left ventricular pressure, aortic pressure, or pulmonary artery pressure.

The pressure measurement module 736 is further adapted to process signals obtained from the pressure sensor 718 to derive pressure measures of interest. The pressure measurement is provided to the therapy module 740, where it is further processed to determine if stimulation therapy may be improved to provide a more desirable outcome. For example, different measures of intrathoracic pressure may be obtained for different stimulation therapies, each defined by a different set of stimulation parameter values, to determine which set of stimulation parameters provides the best measure of intrathoracic pressure. In another example, the measure of intrathoracic pressure may be compared to a predetermine threshold value, to determine if one or more of the stimulation parameters should be adjusted in an attempt to obtain, or at least more closely approach, the threshold value.

Regarding the processing of signals from a motion sensor 720, the pressure signal module 730 may include a diaphragm motion and heart sounds analysis module 738 for analyzing one or more of motion of the diaphragm and sounds associated with the heart. The diaphragm motion and heart sounds analysis module 738 is adapted to receive signals from the motion sensor 720 and to detect a pressure event of interest. As previously described, the motion sensor 720 may be an accelerometer configured to be positioned on or near a diaphragm to sense motion of the diaphragm. The motion sensor 720 may also be an accelerometer or an acoustic transducer configured to be positioned within the patient to sense sounds associated with cardiac function, and to output electrical signals representative of such sounds. Alternatively, the motion sensor 720 may be an impedance/conductance sensor in the form of a pair of electrodes configured to be positioned in or on the diaphragm.

Regarding the therapy module 740, it includes a cardiac-event analysis module 742, a pressure analysis module 744, and a pulse generator 746. The pulse generator 746 is configured to output stimulation therapy to the first ADS therapy delivery mechanism 100a and the second ADS therapy delivery mechanism 100b. The stimulation therapy may be in the form of electrical stimulation, in which case the therapy may be delivered through electrodes 722a, 722b, 724a, 724b.

The stimulation therapy output by the pulse generator 746 is defined by one or more stimulation parameters. For electrical stimulation, the parameters may include: 1) one or more pulse parameters having a value or setting selected to define a stimulation pulse that induces a transient, partial contraction of the diaphragm, and 2) a timing parameter that controls the timing of the delivery of one or more stimulation pulses. The pulse parameters may include, for example, a pulse waveform type, a pulse amplitude, a pulse duration, and a pulse polarity. The timing parameter may include one or more offset periods or delay periods that define a time between a detected cardiac event and a delivery of an electrical stimulation pulse.

One or more of the stimulation parameters, including timing parameters and pulse parameters, may be adjusted by the therapy module 740. With respect to timing parameters, as previously mentioned, the rate of electrical stimulation may be adjusted in response to changes in the heart rate of the patient. Accordingly, the rate of delivery of electrical stimulation pulses may range, for example, between 30 pulses per minute (ppm) and 180 ppm, with a typical rate being around 60 ppm. Likewise, a delay period between a detected cardiac event and a delivery of an electrical stimulation pulse may be adjusted based on a running average of time intervals between detected cardiac events. Regarding pulse parameters, the pulse amplitude may be set to a value between 0.0 volts and 7.5 volts, and the pulse width may be set to a value between 0.0 milliseconds and 1.5 milliseconds. The amplitude may be adjusted, for example, in increments of between 0.1 to 0.5 volts, while the pulse width may be adjusted in increments of between 0.1 to 1.5 milliseconds. The polarity may be changed between a positive polarity and a negative polarity, and the waveform type may be changed from mono-phasic to biphasic, or from a square to a triangular, sinusoidal or sawtooth waveform.

The cardiac-event analysis module 742 is configured to receive cardiac-event information from the cardiac signal module 728 and to process the information to determine the timing parameter. To this end, in one configuration, the cardiac-event analysis module 742 determines a time, relative to a detected cardiac event, at which to deliver a stimulation pulse to the diaphragm. The determined time, referred to as a delay period, may be selected so that the stimulation pulse is delivered just prior to the next expected occurrence of the cardiac event.

The offset periods or delay periods may be based on the time between successive detected cardiac events. For example, the EGM analysis module 732 of the cardiac signal module 728 may be configured to detect ventricular events, e.g., R waves, and to output such detections to the therapy module 740. The cardiac-event analysis module 742 may process the detected ventricular events to determine a statistical measure of time between a number of pairs of successive ventricular events. The cardiac-event analysis module 742 may then determine one or more offset periods or delay periods based on the statistical measure, and control the pulse generator 746 to output stimulation pulses based on the determined offset period or delay period.

The pressure analysis module 744 of the therapy module 740 is configured to receive pressure information, including one or more of a measure of interest, e.g., a pressure measurement, or an event of interest, e.g., end inspiration of a respiration cycle, from the pressure signal module 730. The pressure analysis module 744 is further configured to process the received pressure information to determine if an adjustment of a stimulation parameter is warranted.

In one configuration, the pressure analysis module 744 may receive pressure information corresponding to a measure of interest, and may evaluate the measure of interest against a baseline measure of interest. For example, the received measure of interest may be a measure of an intrathoracic pressure, RA pressure, RV pressure, Ao pressure, or LV pressure at a fiducial point. The pressure analysis module 744 may compare the received measure of interest to the baseline to determine if the comparison outcome is acceptable. If the comparison outcome is not acceptable, the therapy module 740 may adjust one or more stimulation parameters for future stimulation therapy to eventually arrive at a stimulation therapy that results in an acceptable outcome.

In another configuration, the pressure analysis module 744 may receive pressure information corresponding to an occurrence of a pressure event of interest. The pressure event of interest may, for example, relate to respiration cycles of a patient and may be a point of end inspiration within a respiration cycle. In response to the receipt of such pressure information, the pressure analysis module 744 may determine to withhold stimulation therapy or to change one or more stimulation parameters.

The controller 702 includes a memory subsystem 748. The memory subsystem 748 is coupled to the cardiac signal module 728 and the pressure signal module 730, and may receive and store data representative of sensed EGMs, sensed intrathoracic cavity pressure, heart sounds, and sensed cardiovascular pressures, e.g., right ventricular pressures, left ventricular pressure, right atrial pressure, and aortic pressure. The memory subsystem 748 is also coupled to the therapy module 740 and may receive and store data representative of delivered stimulation therapies, including their associated sets of stimulation parameters and times of delivery.

The controller 702 also includes a communication subsystem 750 that enables communication between the controller and other components. These other components may form part of the IMD 700, such as a separately implanted pressure sensor within the intrathoracic cavity, may be separate from the IMD, such as an external programmer used by a physician to program the IMD. The communication subsystem 750 may include a telemetry coil enabling transmission and reception of signals, to or from an external apparatus, via inductive coupling. Alternative embodiments of the communication subsystem 750 could use an antenna for an RF link, or a series of low amplitude high frequency electrical pulses emitted by the sensor that do not illicit muscle or nervous activation, detected by sensing electrodes of the stimulating IMD. The controller 702 also includes a power supply 752 that supplies the voltages and currents necessary for each module of the controller, and a clock supply 754 that supplies the modules with any clock and timing signals.

Regarding the physical structure of the IMD 700, while the foregoing functional description of the IMD describes separate pairs of electrodes 712, 714 and 722a, 724a and 722b, 724b respectively associated with the cardiac event source 706, the first ADS therapy delivery mechanism 100a, and the second ADS therapy delivery mechanism 100b, a configuration of the IMD may include a pair of electrodes configured to perform dual functions. That is, the IMD 700 may include a pair of electrodes configured to both sense cardiac electrical activity and to deliver electrical stimulation. In this configuration, the controller 702 may include an electrode interface that is configured to switch the connection of the electrodes between the cardiac event source 706 and one of the ADS therapy delivery mechanisms 100a, 100b as needed. The electrode interface may also provide other features, capabilities, or aspects, including but not limited to amplification, isolation, and charge-balancing functions, that are required for a proper interface between the electrodes and diaphragm tissue.

Similarly, the respective functions of the separate motion sensors 716, 720 referenced with respect to the cardiac event source 706 and the pressure measurement source 708 may be provided by a single motion sensor shared by the different sources. In this configuration, the controller 702 may include sensor interface that is configured to switch the connection of the single sensor between the cardiac event source 706 and the pressure measurement source 708 if needed. The sensor interface may also provide other features, capabilities, or aspects, including but not limited to amplification and isolation, that are required for a proper interface between the sensor and diaphragm tissue.

The cardiac signal module 728, the pressure signal module 730, and the therapy module 740 of the IMD 700 include or are associated with one or more processors configured to access and execute computer-executable instructions stored in memory associated with the modules. The one or more processors may include a central processor of the controller 702 that executes instructions for all modules 728, 730, 740. Alternatively, each of the various modules 728, 730, 740 may have a dedicated processor. Instructions executed by the one or more processor may be stored in the memory subsystem 748 or in one or more additional memory components (not shown) of the controller 702.

The one or more processors of the controller 702 may be implemented as appropriate in hardware, software, firmware, or combinations thereof. Software or firmware implementations of the one or more processor of the controller 702 may include computer-executable or machine-executable instructions written in any suitable programming language to perform the various functions described herein. The one or more processors of the controller 702 may include, without limitation, a central processing unit (CPU), a digital signal processor (DSP), a reduced instruction set computer (RISC) processor, a complex instruction set computer (CISC) processor, a microprocessor, a microcontroller, a field programmable gate array (FPGA), a System-on-a-Chip (SOC), or any combination thereof. The IMD 700 may also include a chipset (not shown) for controlling communications between the one or more processors of the controller 702 and one or more of the other components of the IMD 700. The one or more processors of the controller 702 may also include one or more application-specific integrated circuits (ASICs) or application-specific standard products (ASSPs) for handling specific data processing functions or tasks.

The memory subsystem 748 and any other memory components of the IMD 700 may include, but is not limited to, random access memory (RAM), flash RAM, magnetic media storage, optical media storage, and so forth. The memory subsystem 748 and other memory components may include volatile memory configured to store information when supplied with power and/or non-volatile memory configured to store information even when not supplied with power. The memory subsystem 748 and other memory components of may store various program modules, application programs, and so forth that may include computer-executable instructions that upon execution by the one or more processors of the controller 702 may cause various operations to be performed. The memory subsystem 748 and other memory components may further store a variety of data manipulated and/or generated during execution of computer-executable instructions by the various modules 728, 730, 740 of the controller 702.

As previously described, the IMD 700 may further include a communication subsystem 750 that may facilitate communication between the IMD 700 and one or more other devices using any suitable communications standard. For example, a LAN interface may implement protocols and/or algorithms that comply with various communication standards of the Institute of Electrical and Electronics Engineers (IEEE), such as IEEE 802.11, while a cellular network interface implement protocols and/or algorithms that comply with various communication standards of the Third Generation Partnership Project (3GPP) and 3GPP2, such as 3G and 4G (Long Term Evolution), and of the Next Generation Mobile Networks (NGMN) Alliance, such as 5G.

The memory subsystem 748 and other memory components may store various program modules, algorithms, and so forth that may include computer-executable instructions that upon execution by the one or more processors associated with the various modules 728, 730, 740 of the controller 702 may cause the various operations of these modules, as described above and further below, to be performed. To this end, the memory subsystem 748 and other memory components store computer-executable instructions that enable a processor to perform: 1) the EGM analysis and heart motion analysis operations of the cardiac signal module 728, 2) the pressure measurement and diaphragm motion / heart sounds analysis operations of the pressure signal module 730, and 3) the cardiac-event analysis, pressure analysis, and EMG analysis of the therapy module 740.

The memory subsystem 748 and other memory components also store computer-executable instructions that enable a processor associated with the therapy module 740 to perform the signal processing, analysis, and ADS therapy delivery associated with each of: 1) the dual-pulse ADS therapy disclosed further below, 2) the paired-pulse ADS therapy disclosed further below, 3) multiple pulse ADS therapy disclosed further below, and 4) the EGM monitoring and health assessment disclosed further below.

The various modules 728, 730, 740 of the controller 702 may be implemented in hardware or software that is executed on a hardware platform. The hardware or hardware platform may be a general purpose processor, a digital signal processor (DSP), an application specific integrated circuit (ASIC), a field programmable gate array (FPGA) or other programmable logic component, discrete gate or transistor logic, discrete hardware components, or any combination thereof, or any other suitable component designed to perform the functions described herein. A general-purpose processor may be a microprocessor, but in the alternative, the processor may be any conventional processor, controller, microcontroller, or state machine. A processor may also be implemented as a combination of computing components, e.g., a combination of a DSP and a microprocessor, a plurality of microprocessors, one or more microprocessors in conjunction with a DSP, or any other such configuration.

Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. The software may reside on a computer-readable medium. A computer-readable medium may include, by way of example, a smart card, a flash memory device (e.g., card, stick, key drive), random access memory (RAM), read only memory (ROM), programmable ROM (PROM), erasable PROM (EPROM), electrically erasable PROM (EEPROM), a general register, or any other suitable non-transitory medium for storing software.

### Bi-Hemispheric Synchronized ADS Therapy

Bi-hemispheric synchronized ADS therapy is a therapy where two, asymptomatic stimulation pulses are delivered to the diaphragm per cardiac cycle, with a first pulse delivered to the right hemisphere of the diaphragm and the second pulse delivered to the left hemisphere. Bi-hemispheric synchronized ADS therapy provides, on a cardiac cycle basis, each of a transient, partial contraction of the left hemisphere and a transient, partial contraction of the right hemisphere. This translates into an intrathoracic pressure modulation most favorable for the optimization of preload and afterload in support of the patient’s hemodynamics. As previously mentioned, a “transient” contraction of the diaphragm is a short, twitching, caudal followed by cranial motion of the diaphragm that lasts in the range of 60 to 180 msec., and is typically about 100 msec. A “partial” contraction of the diaphragm is the part of the diaphragm (less than the entirety of the diaphragm) that exhibits a “transient” contraction.

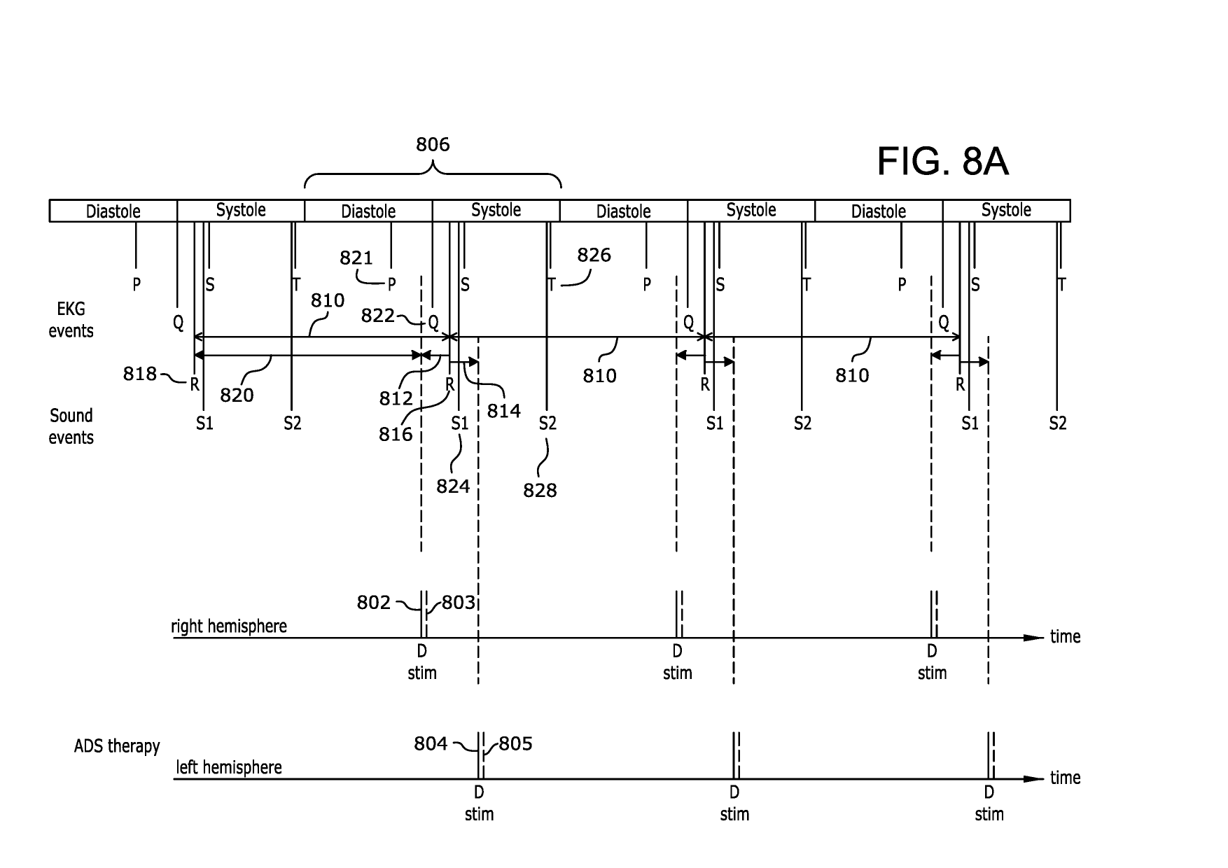

With reference to FIGS. 8A and 8B, in one embodiment, at least two diaphragmatic stimulation pulses 802, 804 are delivered per cardiac cycle 806. The delivery of each stimulation pulse 802, 804 is synchronized to a cardiac event so that a first stimulation pulse 802 is delivered to the right hemisphere in the latter part of diastole (preload benefit) of the cardiac cycle 806 and a second stimulation pulse 804 is delivered to the left hemisphere at the early part of systole (afterload benefit) of the cardiac cycle.

As shown in FIG. 8B, the first stimulation pulse 802 causes a corresponding transient, partial contraction 808 of the right hemisphere of the diaphragm that comprises a movement in the caudal direction followed by a movement in the cranial direction. Similarly, the second stimulation pulse 804 causes a corresponding transient, partial contraction 809 of the left hemisphere of the diaphragm that comprises a movement in the caudal direction followed by a movement in the cranial direction.

Referring to FIG. 8A, “the latter part of diastole” may be the atrial filling window (between a P wave and following Q point), with the corresponding first stimulation pulse 802 preferably being delivered closer to the Q point than the P wave. The “early part of systole” may be the systolic output (between a S1 heart sound and following S2 heart sound), with the corresponding second stimulation pulse 804 preferably being delivered at an offset of about 50-100 msec. after the mitral valve component of the S1 heart sound.

The triggering of the first and second stimulation pulses 802, 804 may be timed relative to a sensed occurrence of a valid cardiac event. In one embodiment, delivery of each of the first and second stimulation pulses 802, 804 is triggered by the same sensed occurrence of a cardiac event. In another embodiment, one of the two stimulation pulses 802, 804 is triggered by a sensed occurrence of a valid cardiac event and the other of the two stimulation pulses 802, 804 is triggered by a sensed occurrence of another cardiac event different from the one that triggered the other stimulation pulse.

A valid cardiac event may be a valid ventricular event (V-event) or a valid atrial event (A-event). The sensed occurrence of a valid cardiac event may correspond to an electrical cardiac event or a mechanical cardiac event. Electrical cardiac events may be a feature of an ECG, such as a P wave, a QRS complex, a Q point of a QRS complex, a R wave, a S point of a QRS complex or a T wave. Mechanical cardiac events may be a S1 heart sound, a S2 heart sound, a S3 sound or a S4 heart sound.

A valid V-event may be an electrical or mechanical event of a ventricle sensed either electrically or mechanically by an IMD. In one configuration, a valid electrical V-event may be an intrinsic depolarization of the ventricle that results from normal electrical conduction through the atrioventricular (AV) node. In electrocardiogram (ECG) terminology, such a valid electrical V-event may be a normal R wave, a normal QRS complex, or a normal T wave. In another configuration, a valid electrical V-event may be a ventricular pacing stimulus delivered to the ventricle and sensed by the IMD. In yet another configuration, a valid electrical V-event may be an evoked response of the ventricle sensed by the IMD. In this regard, an evoked ventricular event corresponds to an electrical depolarization of the ventricle that results from the delivery of a ventricular pacing stimulus.

Associated with a ventricular depolarization, whether intrinsic or evoked, is a physical contraction of the ventricle. Accordingly, each of an intrinsic ventricular depolarization and an evoked ventricular depolarization may be sensed by a mechanical sensor, e.g., in the form of an S1 heart sound or an S2 heart sound. In some cases, the IMD may be programmed to consider what would otherwise be a valid V-event, as a non-valid V-event if that V-event is associated with a non-normal cardiac episode. For example, if the otherwise valid V-event occurs during an episode of ventricular fibrillation or is followed by a premature ventricular contraction, the IMD may deem that V-event non-valid for purposes of delivering diaphragmatic stimulation.

A valid A-event may be an electrical event of an atrium, sensed either electrically or mechanically by an IMD. In one configuration, a valid electrical A-event may be an intrinsic depolarization of the atrium that originates from the sinoatrial (SA) node. In ECG terminology, such a valid electrical A-event may be a normal P wave. In another configuration, a valid electrical A-event may be an atrial pacing stimulus delivered to the atrium and sensed by the IMD. In yet another configuration, a valid electrical A-event may be an evoked response of the atrium sensed by the IMD. In this regard, an evoked atrial event corresponds to an electrical depolarization of the atrium that results from the delivery of an atrial pacing stimulus.

Associated with atrial depolarization, whether intrinsic or evoked, is a physical contraction of the atrium. Accordingly, each of an intrinsic atrial depolarization and an evoked atrial depolarization may be sensed by a mechanical sensor, e.g., in the form of an S4 heart sound. In some cases, the IMD may be programmed to consider what would otherwise be a valid A-event, as a non-valid A-event if that A-event is associated with a non-normal cardiac episode. For example, if the otherwise valid A-event occurs during an episode of atrial tachycardia, atrial fibrillation, or atrial flutter, the IMD may deem that A-event non-valid for purposes of delivering diaphragmatic stimulation.

With reference to FIG. 8A, the triggering of the first and second stimulation pulses 802, 804 is timed relative to the same sensed occurrence of an intrinsic R-wave, with the first stimulation pulse 802 delivered in accordance with a calculated negative offset from the R-wave for the late diastolic pulse, and the second stimulation pulse 804 delivered in accordance with a calculated positive offset to be in the early systolic part. The calculation could be a either a value determined and programmed by the clinician or an actual calculation performed by an IMD 700 based on time intervals between ECG and/or heart sound signal fiducial points.

Regarding calculations performed by an IMD 700, in accordance with embodiments disclosed herein, the timings of the delivery of stimulation pulses 802, 804 may be determined based on sensed occurrences of cardiac events that occur over a number of cardiac cycles 806. For example, a VV interval 810 may be measured over a number of cardiac cycle 806 to obtain an average VV interval. Based on this average VV interval the IMD 700 may calculate a set of offset periods, including a first offset period 812 that defines when a first stimulation pulses 802 is delivered relative to a cardiac event 816, and a second offset period 814 that defines when a second stimulation pulses 804 is delivered relative to the cardiac event 816. The first offset period 812 is characterized by a negative offset relative to the cardiac event 816 and may be described as an “early” or “anticipatory” stimulation in that it occurs before the cardiac event. The second offset period 814 is characterized by a positive offset relative to the cardiac event 816 and may be described as a “late” stimulation in that it occurs after the cardiac event.

The value of the first offset period 812 is selected so that the first stimulation pulse 802 is delivered during late diastole. To this end, the IMD 700 may sense occurrences of other cardiac events to determine a value that results in such placement. For example, with reference to FIG. 8A, the IMD 700 may sense P waves 821 and Q points 822 over the same number of cardiac cycles 806 that it senses R waves 816. Based on the respective timing differences between the P wave 821 and R wave 816 and the Q point 822 and R wave, over a number of cardiac cycles 806, the IMD 700 may calculate a first offset period 812 that places delivery of the first stimulation pulse 802 somewhere between the P wave 821 and Q point 822 that are prior to the R wave 816. In one embodiment the first stimulation pulse 802 is placed so that it is closer to Q point 822 than it is to the P wave 821.

The value of the second offset period 814 is selected so that the second stimulation pulse 804 is delivered during early systole. To this end, the IMD 700 may sense occurrences of other cardiac events to determine a value that results in such placement. For example, with reference to FIG. 8A, the IMD 700 may sense S1 heart sounds 824 and T waves 826 over the same number of cardiac cycles 806 that it senses R waves 816. Based on the respective timing differences between the S1 heart sound 824 and R wave 816 and the T wave 826 and R wave, over a number of cardiac cycle 806, the IMD 700 may calculate a second offset period 814 that places delivery of the second stimulation pulse 804 somewhere between the S1 heart sound 824 and the T wave 826 that are after the R wave 816. In one embodiment the second stimulation pulse 804 is placed so that it is closer to the S1 heart sound 824 than it is to the T wave 826.

Regarding the first stimulation pulse 802, the delivery of this stimulation pulse is triggered by the sensed occurrence of the previous cardiac event 818. In other words, upon detection of the cardiac event 818, the first stimulation pulse 802 is delivered an offset period 820 after such detection that places the pulse in late diastole. To this end, the IMD 700 may calculate the offset period 820 as the difference between the VV interval 810 and the first offset period 812.

While the foregoing description has focused on the delivery of first and second stimulation pulses 802, 804 timed to occur respectively during diastole and systole based on a detection of the same cardiac event, e.g., an R wave, the delivery of these stimulation pulses may be timed to occur during diastole and systole based on detections of other cardiac events.

In some cases, it may be desirable to simulate the diaphragm based on certain diastole cardiac events, such as an offset of passive left ventricular filling or an onset of atrial filling, the occurrences of which generally coincide with a P wave of an ECG. To this end, the IMD 700 may monitor the time between a P wave 821 and a following Q point 822 over a number of cardiac cycles 806, and calculate an offset from a P wave that places a delivery of a stimulation pulse 802 after the P wave 821 but before the following Q point 822. Once this offset is determined, subsequent detections of these diastole cardiac events by an IMD 700 may trigger a delivery of a stimulation pulse 802 during late diastole.

In some cases, it may be desirable to simulate the diaphragm based on certain systole cardiac events, such as: 1) an onset of electrical systole or an offset of atrial filling, the occurrences of which generally coincide with a Q point 822 of an ECG; 2) an offset of electrical systole, the occurrences of which generally coincide with a T wave 826 of an ECG; 3) an onset of mechanical systole, the occurrences of which generally coincide with a S1 heart sound 824; 4) an offset of mechanical systole or an onset of passive left ventricular filling, the occurrences of which generally coincide with a S2 heart sound 828; 5) an onset of left ventricular systolic output, the occurrences of which generally coincide with a mitral valve component of a S1 heart sound 824; 6) an offset of left ventricular systolic output, the occurrences of which generally coincide with an aortic valve component of a S2 heart sound 828; 7) an onset of right ventricular systolic output, the occurrence of which generally coincide with a tricuspid valve component of a S1 heart sound 824; or 8) an offset of right ventricular systolic output, the occurrences of which generally coincide with a pulmonic valve component of a S2 heart sound 828.

To this end, the IMD 700 may monitor the time between one of a Q point 822 or an S1 heart sound 824 and a following one of a T wave 826 or a S2 heart sound 828 over a number of cardiac cycles 806, and calculate an offset from of a Q point 822 or an S1 heart sound 824 that place a delivery of a second stimulation pulse 804 after the Q point but before the T wave, or after the S1 heart sound but before the S2 heart sound. Once this offset it is determined, subsequent detections of these systole cardiac events by an IMD 700 may trigger a delivery of a second stimulation pulse 804 during early systole.

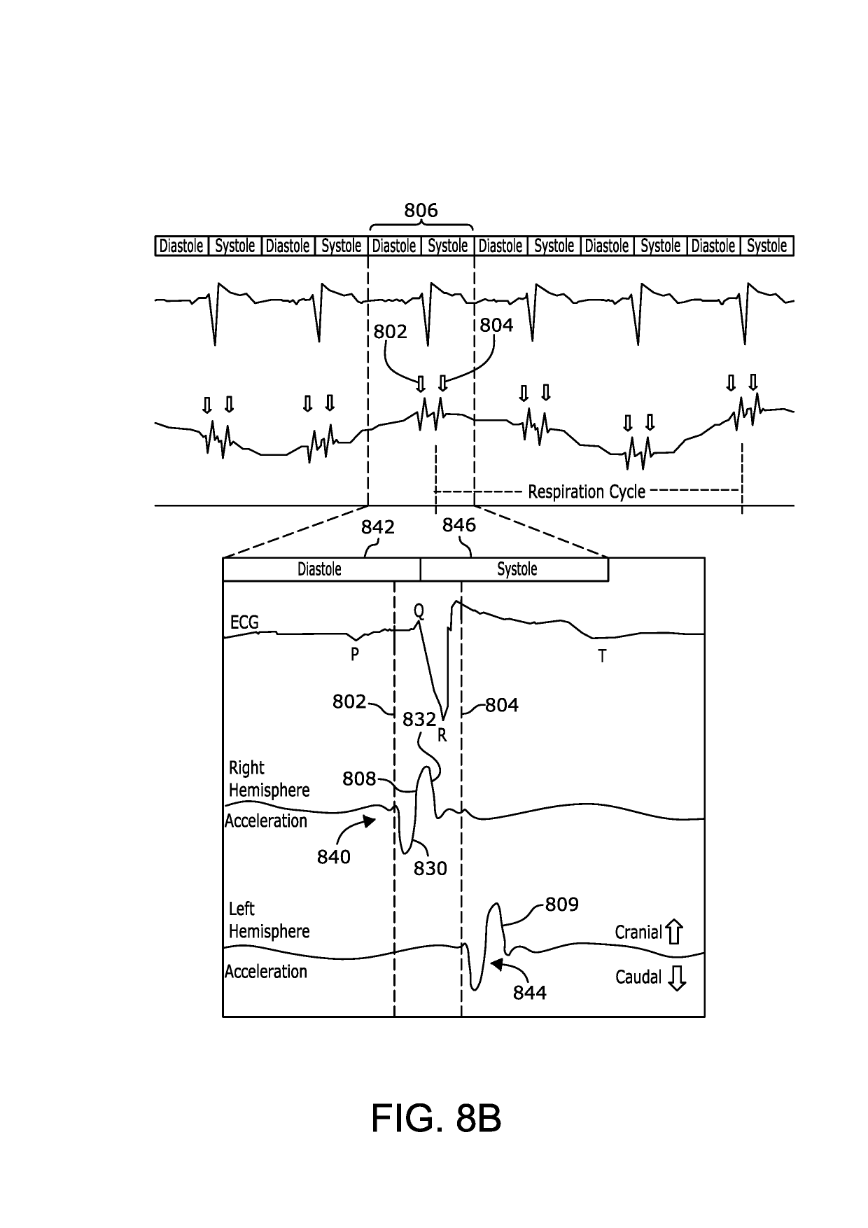

FIG. 9 is a flowchart of a method of affecting cardiac function of a patient. The method may be performed by the IMD 700 of FIG. 7 or a similar apparatus or system. For example, the method may be performed by an apparatus having a first ADS therapy delivery mechanism 100a, e.g., one or more electrodes 722a, 724a, configured for placement on or near the right hemisphere of a diaphragm, a second ADS therapy delivery mechanism 100b, e.g., one or more electrodes 722b, 724b, configured for placement on or near the left hemisphere of a diaphragm, and a controller 702 coupled to first therapy delivery mechanism and the second therapy delivery mechanism. The controller 702 is configured, for example, though executable program instructions stored in a memory, to perform the method described below with reference to FIG. 9.

At block 902, and with additional reference to FIGS. 8A and 8B, a stimulation is delivered to a right hemisphere of a diaphragm of the patient during a diastolic phase of a cardiac cycle of the patient. The stimulation results in an asymptomatic, transient, partial contraction of the right hemisphere of the diaphragm.

In some embodiments, the stimulation to the right hemisphere is a single electrical pulse 802. Delivery of the single stimulation pulse 802 to the right hemisphere may be timed to a cardiac event. To this end, an occurrence of a first cardiac event 818 is detected, and the stimulation pulse 802 is delivered at or near the end of an offset period 820 from the detected first cardiac event. The offset period 820 may be referred to as a diastolic offset period. The diastolic offset period 820 places the delivery of the stimulation pulse 802 at a latter part of diastole of the cardiac cycle 806.

The diastolic offset period 820 may be determined by detecting, over a plurality of cardiac cycles, a time of occurrence of: a) the first cardiac event 818, and b) one of an onset of an atrial event 821, e.g., a detected P wave, and an offset of a first ventricular event 821, e.g., also a detected P wave, and c) an onset of a second ventricular event 822, 824 that follows the onset of the atrial event and the onset of the first ventricular event. The onset of a second ventricular event may correspond to a detected Q point 822 in a QRS complex, or a detected S1 heart sound 824. The respective times of occurrences are processed to calculate a period of time from the first cardiac events 818 to a time in between a) the detected P wave and b) the onset of a second ventricular event (e.g., a detected Q point 822 in a QRS complex, or a detected S1 heart sound 824) that follows the detected P wave. The calculated period of time corresponds to the diastolic offset period 820.

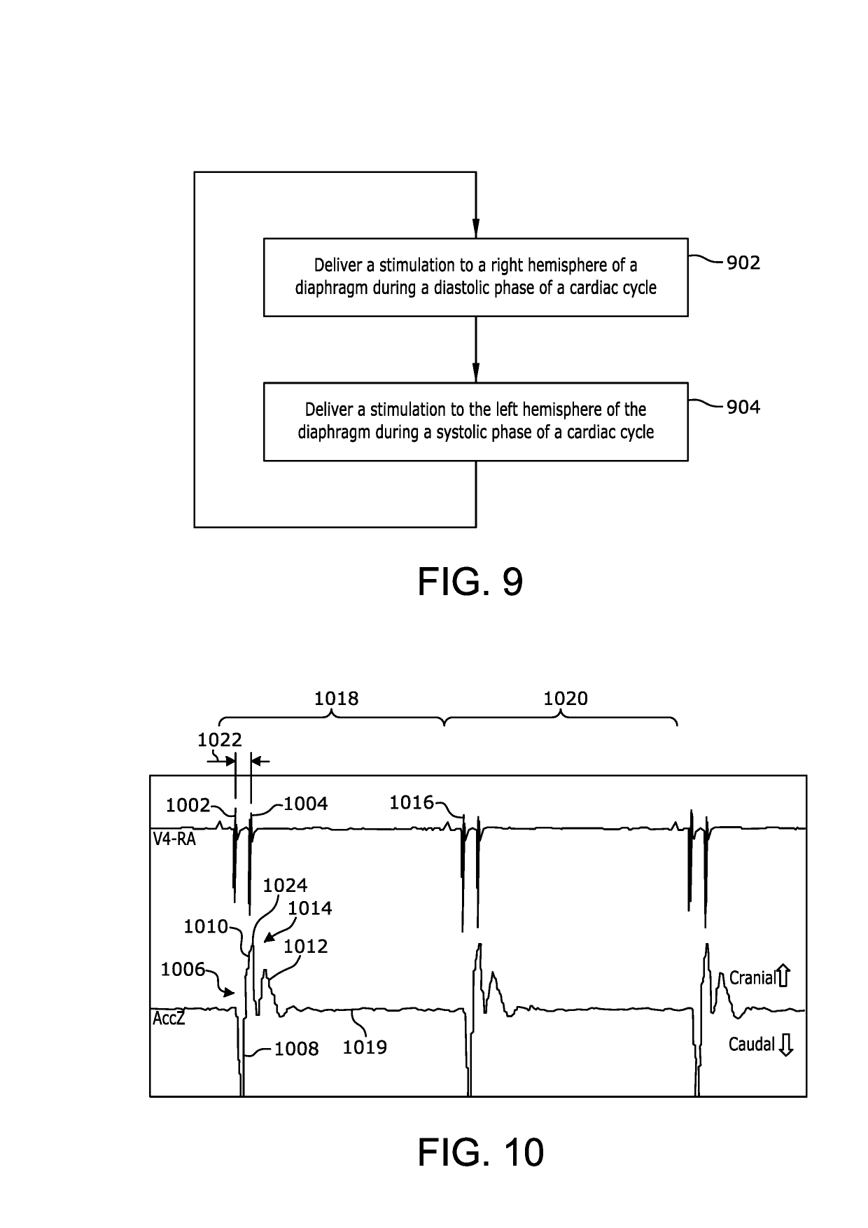

In some embodiments, the stimulation to the right hemisphere can be a pair of right-side stimulation pulses 802, 803 spaced apart between 50-75 msec. With reference to FIG. 10, delivering a pair of ADS pulses 1002, 1004 in this manner to either diaphragm manipulates a transient, partial contraction of that hemisphere in a way that enhances and adds to the duration of one of a caudal phase or a cranial phase of the contraction. For example, in the case of right-side stimulation, it is possible to extend the duration of a cranial phase 1014 of a transient, partial contraction of the right hemisphere of the diaphragm for a brief time through delivery of the second ADS pulse 1004 soon after the delivery of the first ADS pulse 1002, without the extended cranial phase overlapping with the delivery of an ADS pulse 804 to the left hemisphere of the diaphragm. A delivery of an ADS pulse 1004 for purpose of extending a particular phase of contraction is feasible because the refractory period of the diaphragm is short, e.g., between 1 msec. and 4 msec., and nearly independent of heart rate as the diaphragm tone is driven by respiratory needs. In other words, even in cases of increased heart rate, there is sufficient time during a current cardiac cycle 1018 to extend one or both of a cranial phase 1014 of a partial contraction or a caudal phase of a partial contraction, before the next cardiac cycle 1020 initiates and triggers a next ADS pulse 1016.

With continued reference to FIG. 10, an appropriately timed and spaced apart pair of ADS pulses including a first ADS pulse 1002 and a second ADS pulse 1004 may shift the balance of contraction phases to one of predominantly caudal or predominantly cranial to impact intrathoracic pressure and cardiovascular flow. To this end, a portion 202a, 202b or part of the hemisphere 105a, 105b may be stimulated as it is moving in either of the caudal phase or the cranial phase of a contraction. By stimulating the portion 202a, 202b of the hemisphere 105a, 105b prior to that portion completing its transient contraction, the morphology of the mechanical response of the portion can be altered in a way to shift the balance of the phases of the transient contraction between one that is predominantly caudal, i.e., the portion 202a, 202b of the hemisphere 105a, 105b moves in the caudal direction for a period of time greater than the time the part moves in the cranial direction, and predominantly cranial, i.e., the portion 202a, 202b of the hemisphere 105a, 105b moves in the cranial direction for a period of time greater than the time the part moves in the caudal direction.

In one embodiment, a primary function and benefit of second ADS pulse 1004 is to shorten the first cranial phase initiated by the first ADS pulse 1002 as it might come too early during a cardiac cycle 1018. The second ADS pulse 1004 cuts that first cranial phase short and creates a second cranial phase later in the cardiac cycle 1018 when there is hemodynamic benefit achieved by increasing the pressure on the heart/vessels. An illustration of a shift in diaphragm acceleration resulting from the delivery of a pair of appropriately timed consecutive ADS pulses 1002, 1004 is shown in FIG. 10. The first ADS pulse 1002 results in a typical caudal-followed-by-cranial transient, partial contraction 1006 or twitch of the portion 202a, 202b of the hemisphere 105a, 105b having a caudal phase 1008 followed by the beginning of a first cranial portion 1010 of a cranial phase 1014. During the first cranial portion 1010 of the transient, partial contraction, a second ADS pulse 1004 is delivered. For example, the timing of delivery of the second ADS pulse 1004 may be based on the typical time for the portion 202a, 202b of the hemisphere 105a, 105bto complete a transient, partial contraction. This contraction time is typically between 60 to 180 msec., with the caudal phase taking about half the total time and the cranial phase taking half the total time. Accordingly, an appropriately timed second ADS pulse 1004 for purposes of extending a cranial phase 1014 may be delivered somewhere between 50-75 msec. after the delivery of the first ADS pulse 1002.

Delivery of the second ADS pulse 1004 at this time overlaps with the effect of the first ADS pulse 1002 and induces a sharp change, e.g., reduction, in the acceleration of the diaphragm in the cranial direction. This change in acceleration manifests graphically in FIG. 10 as a narrow-width first cranial portion 1010 having a short, near-vertical drop downward to the baseline 1019. Subsequent to this reduction in acceleration, the second ADS pulse 1004 induces an increase in acceleration of the diaphragm in the cranial direction, followed by a reduction in the acceleration of the diaphragm in the cranial direction. These changes in acceleration manifests graphically in FIG. 10 as a second cranial portion 1012. The second cranial portion 1012, together with the first cranial portion 1010 produce an overall diaphragmatic contraction that is predominately cranial. In other words, the balance of the overall diaphragm movement is shifted to the cranial phase. This is beneficial because movement of the diaphragm in the cranial direction results in an increase in intrathoracic pressure, which in turn increases the pressure on the heart and vessels in a way that it augments cardiac output if timed correctly to the beginning of systole.

Returning to FIG. 9, at block 904, and with additional reference to FIGS. 8A and 8B, a stimulation is delivered to the left hemisphere of the diaphragm during a systolic phase of the cardiac cycle. The stimulation results in an asymptomatic, transient, partial contraction of the left hemisphere of the diaphragm.

In some embodiments, the stimulation to the left hemisphere can be a single electrical pulse 804. Delivery of the stimulation pulse 804 may also be timed to a cardiac event. To this end, an occurrence of a second cardiac event 816 is detected, and the stimulation pulse 804 is delivered at or near the end of an offset period 814 from the detected second cardiac event 816. The offset period 814 may be referred to as a systolic offset period. The systolic offset period 814 places the delivery of the stimulation pulse 804 at an early part of systole of the cardiac cycle 806.

The systolic offset period 814 may be determined by detecting, over a plurality of cardiac cycles, a time of occurrence of: a) the second cardiac event 816, and b) one of an onset of electrical systole 822, e.g., a detected Q point, and an onset of mechanical systole 824, e.g., a detected S1 heart sound, and c) one of an offset of electrical systole 826, e.g., a detected T wave, and an offset of mechanical systole 828, e.g., a detected S2 heart sound. The respective times of occurrences are processed to calculate a period of time from the second cardiac events 816 to a time in between: a) either of the detected Q point 822 or the detected S1 heart sound 824, and b) either of the detected T wave 826 or the detected S2 heart sound 828. The calculated period of time corresponds to the systolic offset period 814.

In some embodiments, the stimulation to the left hemisphere can be a pair of electrical pulses 804, 805 spaced apart between 50-75 msec. With reference to FIG. 10, In this case, it is possible to extend the duration of a cranial phase 1014 of a transient, partial contraction of the left hemisphere of the diaphragm for a brief time through delivery of the second ADS pulse 1004 soon after the delivery of the first ADS pulse 1002, without the extended cranial phase overlapping with the delivery of the next ADS pulse 802 to the right hemisphere of the diaphragm.

While in the foregoing description, the second cardiac event 816 in FIG. 8A that triggers delivery of the stimulation 804 to the left hemisphere, also referred to as the left-side stimulation, is different from the first cardiac event 818 that triggers delivery of the stimulation 802 to the right hemisphere, also referred to as the right-side stimulation, the first cardiac event and the second cardiac event may be the same event within a same cardiac cycle. For example, delivery of each of the right-side stimulation 802 and the left-side stimulation 804 may be timed to a detection of a P wave 821.

In other embodiments, the first cardiac event 818 that triggers delivery of a right-side stimulation pulse 802 and the second cardiac event 816 that triggers delivery of a left-side stimulation pulse 804, may be the same cardiac event type in consecutive cardiac cycles. In the example of FIG. 8A, the first cardiac event 818 is a detected R wave from a prior cardiac cycle that triggers the right-side stimulation pulse 802 in the current cardiac cycle 806, while the second cardiac event 816 is a detected R wave in the current cardiac cycle that triggers the left-side stimulation pulse 804 in the current cardiac cycle 806. While the first and second cardiac events are electrical events, e.g., R waves, the first and second cardiac events may be mechanical events, e.g., an S1 heart sound.

In other embodiments, the first cardiac event that triggers delivery of a right-side stimulation pulse 802 and the second cardiac event that triggers delivery of a left-side stimulation pulse 804, may be different cardiac events in a same cardiac cycle. For example, with reference to FIG. 8A, the first cardiac event may be a detected P wave 821 in the current cardiac cycle 806, while the second cardiac event 816 may be a detected R wave in the current cardiac event. Again, while the first and second cardiac events may be electrical events, e.g., P wave, and R wave, one or more of the first and second cardiac events may be a mechanical event, e.g., an S1 heart sound.

The various aspects of this disclosure are provided to enable one of ordinary skill in the art to practice the present invention. Various modifications to exemplary embodiments presented throughout this disclosure will be readily apparent to those skilled in the art, and the concepts disclosed herein may be extended to other magnetic storage devices. Thus, the claims are not intended to be limited to the various aspects of this disclosure, but are to be accorded the full scope consistent with the language of the claims.

What is claimed is:

1. An apparatus for bi-hemispheric stimulation of a diaphragm, the apparatus comprising:
a first therapy delivery mechanism configured for placement on or near a right hemisphere of a diaphragm;
a second therapy delivery mechanism configured for placement on or near a left hemisphere of the diaphragm; and
a controller coupled to the first therapy delivery mechanism and the second therapy delivery mechanism, and configured to:
deliver through the first therapy delivery mechanism, a first stimulation to the right hemisphere of the diaphragm during a diastolic phase of a cardiac cycle, wherein the stimulation is configured to produce a singular, transient, partial contraction of the right hemisphere of the diaphragm; and
deliver through the first therapy delivery mechanism, a second stimulation to the left hemisphere of the diaphragm during a systolic phase of the cardiac cycle, wherein the stimulation is configured to produce a singular, transient, partial contraction of the left hemisphere of the diaphragm.

2. The apparatus of claim 1, wherein the stimulation to the right hemisphere is a single electrical pulse.

3. The apparatus of claim 1, wherein the stimulation to the right hemisphere is a pair of electrical pulses spaced apart between 50 and 75 milliseconds.

4. The apparatus of claim 1, wherein the stimulation to the left hemisphere is a single electrical pulse.

5. The apparatus of claim 1, wherein the stimulation to the left hemisphere is a pair of electrical pulses spaced apart between 50 and 75 milliseconds.

6. The apparatus of claim 1, wherein:
the controller delivers the first stimulation by being further configured to:
detect an occurrence of a first cardiac event, and
deliver the first stimulation at or near the end of a diastolic offset period from the detected occurrence of a first cardiac event that places the delivery of the first stimulation at a latter part of diastole of the cardiac cycle; and
the controller delivers the second stimulation by being further configured to:
detect an occurrence of a second cardiac event, and
deliver the second stimulation at or near the end of a systolic offset period from the detected occurrence of a second cardiac event that places the delivery of the second stimulation at an early part of systole of the cardiac cycle.

7. The apparatus of claim 6, wherein the first cardiac event and the second cardiac event are a same cardiac event type in consecutive cardiac cycles, the same cardiac event type corresponding to one of an electrical event and a mechanical event.

8. The apparatus of claim 6, wherein the first cardiac event and the second cardiac event are different cardiac events in a same cardiac cycle, each of the different cardiac events corresponding to one of an electrical event and a mechanical event.

9. The apparatus of claim 6, further comprising one or more electrodes configured for placement on or near a heart, wherein the controller is configured to detect at least one of the occurrence of the first cardiac event and the occurrence of the second cardiac event based on signals sensed by the one or more electrodes.

10. The apparatus of claim 6, further comprising a motion sensor configured for placement on or near the diaphragm or on or near a heart, wherein the controller is configured to detect at least one of the occurrence of the first cardiac event and the occurrence of the second cardiac event based on signals sensed by the motion sensor.

11. A method of affecting cardiac function of a patient, the method comprising:
delivering a stimulation to a right hemisphere of a diaphragm of the patient during a diastolic phase of a cardiac cycle of the patient, wherein the stimulation results in a singular, asymptomatic, transient, partial contraction of the right hemisphere of the diaphragm; and
delivering a stimulation to a left hemisphere of the diaphragm during a systolic phase of the cardiac cycle, wherein the stimulation results in a singular, asymptomatic, transient, partial contraction of the left hemisphere of the diaphragm.

12. The method of claim 11, wherein the stimulation to the right hemisphere is a single electrical pulse.

13. The method of claim 11, wherein the stimulation to the right hemisphere is a pair of electrical pulses spaced apart between 50-75 milliseconds.

14. The method of claim 11, wherein the stimulation to the left hemisphere is a single electrical pulse.

15. The method of claim 11, wherein the stimulation to the left hemisphere is a pair of electrical pulses spaced apart between 50-75 milliseconds.

16. The method of claim 11, wherein delivering the stimulation to the right hemisphere comprises:
detecting an occurrence of a first cardiac event; and
delivering the stimulation at or near the end of an offset period from the detected occurrence of a first cardiac event, which offset period places the delivery of the stimulation at a latter part of diastole of the cardiac cycle.

17. The method of claim 11, wherein delivering the stimulation to the left hemisphere comprises:
detecting an occurrence of a second cardiac event; and
delivering the stimulation at or near the end of an offset period from the detected occurrence of a second cardiac event, which offset period places the delivery of the stimulation at an early part of systole of the cardiac cycle.
